(19) United States
(12) Patent Application Publication
(10) Pub. No.: US 2024/0271175 A1
(43) Pub. Date: Aug. 15, 2024

(54) LEADER PEPTIDES AND POLYNUCLEOTIDES ENCODING THE SAME

(71) Applicant: Novozymes A/S, Bagsvaerd (DK)

(72) Inventors: Aki Tomiki-Hashizume, Chiba-shi (JP); Hiroshi Teramoto, Chiba (JP)

(73) Assignee: Novozymes A/s, Bagsvaerd (DK)

(21) Appl. No.: 18/251,533

(22) PCT Filed: Nov. 2, 2021
(86) PCT No.: PCT/EP2021/080303

§ 371 Date: May 2, 2023

(30) Foreign Application Priority Data
Nov. 2, 2020 (DK) PA202001235

(51) Int. Cl. C12P 21/02; C07K 7/06; C12N 1/14; C12N 9/34; C12N 15/62; C12N 15/80; C12R 1/685

(52) U.S. Cl. CPC C12P 21/02; C07K 7/06; C12N 1/14; C12N 9/2428; C12N 15/625; C12N 15/80; C07K 2319/02; C12R 2001/685

(57) ABSTRACT

The present invention relates to leader peptides, leader peptide fusion proteins, signal peptides, polynucleotides encoding the leader peptides and signal peptides, and to nucleic acid constructs, vectors and host cells comprising the polynucleotides as well as methods of producing a polypeptide of interest in host cells expressing the leader peptides in translational fusion with the polypeptide of interest.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to leader peptides, leader peptide fusion proteins, signal peptides, polynucleotides encoding the leader peptides and signal peptides, and to nucleic acid constructs, vectors and host cells comprising the polynucleotides as well as methods of producing a polypeptide of interest in host cells expressing the leader peptides in translational fusion with the polypeptide of interest.

### Description of the Related Art

Recombinant gene expression in fungal or bacterial hosts is a common method for recombinant protein production. Recombinant proteins produced in such host cell systems are enzymes and other valuable proteins. As an example, WO2011127802 describes host cells and methods for producing glucoamylases. In industrial and commercial purposes, the productivity of the applied cell systems, i.e. the production of total protein per fermentation unit, is an important factor of production costs. Traditionally, yield increases have been achieved through mutagenesis and screening for increased production of proteins of interest. However, this approach is mainly only useful for the overproduction of endogenous proteins in isolates containing the enzymes of interest. Therefore, for each new protein or enzyme product, a lengthy strain and process development program is required to achieve improved productivities.

For the overexpression of heterologous proteins in fungal or bacterial host cell systems, the production process is recognized as a complex multi-phase and multi-component process. Cell growth and product formation are determined by a wide range of parameters, including the composition of the culture medium, fermentation pH, fermentation temperature, dissolved oxygen tension, shear stress, and fungal morphology.

Various approaches to improve expression and secretion have been used in fungi and bacteria. For the expression of heterologous genes, codon-optimized, synthetic genes can improve the transcription rate, whereas the overexpression of secretion chaperones is used to protect the heterologous protein from degrading. To obtain high-level expression of a particular gene, a well-established procedure is targeting multiple copies of the recombinant gene constructs to the locus of a highly expressed endogenous gene. A further strategy for improving protein yield is described in WO 2011/075677 (Novozymes A/S) by the disruption of native proteases. Despite the presented approaches, it is of continuous interest to further improve recombinant protein production in fungal and bacterial host cells.

The object of the present invention is to provide a modified host strain and a method of protein production with increased productivity of the recombinant protein.

### SUMMARY OF THE INVENTION

The present invention is based on the surprising and inventive finding that a synthetic leader peptide fused upstream to a heterologous protein can provide an improved expression, activity, and/or yield of the heterologous protein compared to the expression of the heterologous protein in the absence of said leader peptide. Furthermore, the inventors also have surprisingly found that the leader peptide as part of or in combination with different signal peptides can provide improved expression, activity, and/or yield of the heterologous protein.

The identified leader peptides are used in a method of enhancing secretion of recombinant polypeptides produced in host cells, such as fungal host cells. Polynucleotides encoding the novel leader peptides and a method of producing heterologous proteins using said polynucleotides are described. Generally, thermostabilized proteins are more challenging to produce at an industrial scale when compared to their wild type, mostly due to lowered expression levels of the thermostabilized variants. For the protein engineering (PE) of such (stable) variants, low expression levels during fermentation are therefore a major cause for the deselection of engineered protein variant candidates, restricting the PE work significantly. As described in the Examples, the inventors have carried out PE work for a heterologous protein (glucoamylase of AnPav498) resulting in an elongated signal sequence/additional leader peptide (JPO001) with increased expression levels (N=16) and transformation efficiency. Thermostable variants of anPav498 (JPO variants) were developed by PE focusing on the improvement of both performance and yield. JPO051 and JPO124 generated from the backbone molecule (JPO001) improved thermostability and at the same time retained expression level high enough to be used for industrial production of heterologous enzymes. The inventors also have shown that the high expression can be obtained in different strains, different cultivation medium and by fusing the leader peptide to different signal peptides (JSP035 and JSP038). The elongated signal sequence/leader peptide of the present invention can therefore be applied as tool during PE work for the development of protein variants, such as thermostable protein variants. We expect that these findings also apply to other proteins, such as other glycoproteins and in particular to other glucoamylases.

Thus, in a first aspect the present invention relates to a fungal host cell comprising in its genome:
a first polynucleotide encoding a polypeptide of interest; and
a second polynucleotide operably linked in translational fusion to the first polynucleotide upstream of the first polynucleotide, said second polynucleotide encoding a leader peptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO: 2 (FARAPVAAR).

In a second aspect, the present invention relates to a method for producing a polypeptide of interest, the method comprising:
(i) providing a fungal host cell according to the first aspect of the invention,
(ii) cultivating said fungal host cell under conditions conducive for expression of the polypeptide of interest; and, optionally
(iii) recovering the polypeptide of interest.

In a third aspect, the present invention relates to a nucleic acid construct comprising a first polynucleotide encoding a polypeptide of interest, and a second polynucleotide operably linked to the first polynucleotide encoding a leader peptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO: 2 (FARAPVAAR).

In a fourth and final aspect, the present invention relates to an expression vector comprising a nucleic acid construct according to the third aspect.

### DEFINITIONS

In accordance with this detailed description, the following definitions apply. Note that the singular forms “a,” “an,” and “the” include plural references unless the context clearly dictates otherwise.

Reference to “about” a value or parameter herein includes aspects that are directed to that value or parameter per se. For example, description referring to “about X” includes the aspect “X”.

Unless defined otherwise or clearly indicated by context, all technical and scientific terms used herein have the same meaning as commonly understood by one of ordinary skill in the art to which this invention belongs.

Catalytic domain: The term “catalytic domain” means the region of an enzyme containing the catalytic machinery of the enzyme.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon, such as ATG, GTG, or TTG, and ends with a stop codon, such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a polypeptide of the present invention. Each control sequence may be synthetic, native (i.e., from the same gene) or heterologous (i.e., from a different gene) to the polynucleotide encoding the polypeptide or native or heterologous to each other. Such control sequences include, but are not limited to, a leader peptide, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” means any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fusion polypeptide: The term “fusion polypeptide” is a polypeptide in which one polypeptide is fused at the N-terminus or the C-terminus of a polypeptide of the present invention. A fusion polypeptide is produced by fusing a polynucleotide encoding another polypeptide to a polynucleotide of the present invention. Techniques for producing fusion polypeptides are known in the art and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779). A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

Glucoamylase: The term “glucoamylase” means a protein with glucoamylase activity (EC number 3.2.1.3) that catalyzes the hydrolysis of terminal (1->4)-linked alpha-D-glucose residues successively from non-reducing ends of the chains with release of beta-D-glucose. For purposes of the present invention, glucoamylase activity is determined according to the procedure described in the Examples. In one aspect, the polypeptides of the present invention have at least 20%, e.g., at least at least 40%, at least 50%, at least 60%, at least 70%, at least 80%, at least 90%, at least 95%, or at least 100% of the glucoamylase activity of the mature polypeptide of SEQ ID NO: 15, SEQ ID NO: 16, SEQ ID NO: 17, SEQ ID NO: 18, SEQ ID NO: 47, SEQ ID NO: 49, or SEQ ID NO: 51.The term “glucoamylase” is interchangeable with the terms “amyloglucosidase”, “glucan 1,4-α-glucosidase”, and/or “γ-amylase”.

Glycoprotein: The term “glycoprotein” means a conjugated protein in which the non-protein group is a carbohydrate. Glycoproteins contain oligosaccharide chains/glycans covalently attached to polypeptide sidechains. The carbohydrate is attached to the protein during co-translational modification and/or post-translational modification. Glycoproteins can contain N-linked and/or O-linked oligosaccharide residues. Non-limiting examples for a glycoprotein are an alpha-glucosidase, such as the glucoamylases of SEQ ID NO: 15, SEQ ID NO: 16, SEQ ID NO: 17, SEQ ID NO: 18, SEQ ID NO: 47, SEQ ID NO: 49, and SEQ ID NO: 51.

Heterologous: The term “heterologous” means, with respect to a host cell, that a polypeptide or nucleic acid does not naturally occur in the host cell. The term “heterologous” means, with respect to a polypeptide or nucleic acid, that a control sequence, e.g., promoter, or domain of a polypeptide or nucleic acid is not naturally associated with the polypeptide or nucleic acid, i.e., the control sequence is from a gene other than the gene encoding the mature polypeptide of SEQ ID NO: 15, SEQ ID NO: 16, SEQ ID NO: 17, SEQ ID NO: 18, SEQ ID NO:

47, SEQ ID NO: 49, or SEQ ID NO: 51. The term “heterologous” means, with respect to a leader peptide, that the protein of interest and/or the signal peptide is not naturally associated with the leader peptide, i.e., the leader peptide is from a gene other than the gene encoding the mature polypeptide of SEQ ID NO: 15, SEQ ID NO: 16, SEQ ID NO: 17, SEQ ID NO: 18, SEQ ID NO: 47, SEQ ID NO: 49, or SEQ ID NO: 51, and/or that the leader peptide is from a gene other than the gene encoding the signal peptide of SEQ ID NO: 4, SEQ ID NO: 41, or SEQ ID NO: 52.

Host cell: The term “host cell” means any microbial, fungal or plant cell into which a nucleic acid construct or expression vector comprising a polynucleotide of the present invention has been introduced. Methods for introduction include but are not limited to protoplast fusion, transfection, transformation, electroporation, conjugation, and transduction. In some embodiments, the host cell is an isolated recombinant host cell that is partially or completely separated from at least one other component with, including but not limited to, proteins, nucleic acids, cells, etc.

Hybrid polypeptide: The term “hybrid polypeptide” means a polypeptide comprising domains from two or more polypeptides, e.g., an elongated signal peptide module (synthetic or from one polypeptide) and a catalytic domain from another polypeptide. The domains may be fused at the N-terminus or the C-terminus.

Hybridization: The term “hybridization” means the pairing of substantially complementary strands of nucleic acids, using standard Southern blotting procedures. Hybridization may be performed under medium, medium-high, high or very high stringency conditions. Medium stringency conditions means prehybridization and hybridization at 42° C. in 5×SSPE, 0.3% SDS, 200 micrograms/ml sheared and denatured salmon sperm DNA, and 35% formamide for 12 to 24 hours, followed by washing three times each for 15 minutes using 0.2×SSC, 0.2% SDS at 55° C. Medium-high stringency conditions means prehybridization and hybridization at 42° C. in 5×SSPE, 0.3% SDS, 200 micrograms/ml sheared and denatured salmon sperm DNA, and 35% formamide for 12 to 24 hours, followed by washing three times each for 15 minutes using 0.2×SSC, 0.2% SDS at 60° C. High stringency conditions means prehybridization and hybridization at 42° C. in 5×SSPE, 0.3% SDS, 200 micrograms/ml sheared and denatured salmon sperm DNA, and 50% formamide for 12 to 24 hours, followed by washing three times each for 15 minutes using 0.2×SSC, 0.2% SDS at 65° C. Very high stringency conditions means prehybridization and hybridization at 42° C. in 5×SSPE, 0.3% SDS, 200 micrograms/ml sheared and denatured salmon sperm DNA, and 50% formamide for 12 to 24 hours, followed by washing three times each for 15 minutes using 0.2×SSC, 0.2% SDS at 70° C.

Isolated: The term “isolated” means a polypeptide, nucleic acid, cell, or other specified material or component that is separated from at least one other material or component with which it is naturally associated as found in nature, including but not limited to, for example, other proteins, nucleic acids, cells, etc. An isolated polypeptide includes, but is not limited to, a culture broth containing the secreted polypeptide.

Leader peptide: Precursor polypeptides typically consist of an N-terminal leader and a C-terminal core peptide. The precursor peptides are ribosomally synthesized and post-translationally modified to their active structures. The role most commonly proposed for the leader peptides is that of a secretion signal. Successful protein secretion requires effective translocation of the protein across the endoplasmic reticulum-plasma membrane or cell membrane. Proteins destined for secretion are targeted to the membrane via their respective secretion signals that are usually located at the N-terminal of nascent polypeptides. A second role that is frequently postulated is that of a recognition motif for the post-translational modification enzymes. The leader peptide is encoded by a leader sequence which may regulate gene expression at the level of transcription or translation as described by Mølhøj & Dal Degan (Leader sequences are not signal peptides, Nature Biotechnology 22, 1502 (2004)). In the context of the present invention, the leader peptide is cleaved off the polypeptide of interest, leaving a mature polypeptide of interest. In one aspect, a second polynucleotide encoding a leader peptide is operably linked in translational fusion to a first polynucleotide encoding a polypeptide of interest upstream of the first polynucleotide, said leader peptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO: 2 (FARAPVAAR). In a preferred embodiment the leader peptide comprises, consists essentially of, or consists of SEQ ID NO: 2.

Mature polypeptide: The term “mature polypeptide” means a polypeptide in its mature form following N-terminal processing (e.g., removal of signal peptide and/or leader peptide). In one aspect, the mature polypeptide is one of SEQ ID NO: 15, SEQ ID NO: 16, SEQ ID NO: 17 and SEQ ID NO: 18.

Mature polypeptide coding sequence: The term “mature polypeptide coding sequence” means a polynucleotide that encodes a mature polypeptide having biological activity. In one aspect, the mature polypeptide coding sequence is nucleotides 91 to 1878 of SEQ ID NO: 9.

Native: The term “native” means a nucleic acid or polypeptide naturally occurring in a host cell.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Purified: The term “purified” means a nucleic acid or polypeptide that is substantially free from other components as determined by analytical techniques well known in the art (e.g., a purified polypeptide or nucleic acid may form a discrete band in an electrophoretic gel, chromatographic eluate, and/or a media subjected to density gradient centrifugation). A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

Recombinant: The term “recombinant,” when used in reference to a cell, nucleic acid, protein or vector, means that it has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding a polypeptide is a recombinant vector. The term “recombinant” is synonymous with “genetically modified” and “transgenic”.

Sequence identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the no-brief option must be specified in the command line. The output of Needle labeled “longest identity” is calculated as follows:

(Identical Residues×100)/(Length of Alignment−Total Number of Gaps in Alignment)

For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows:

(Identical Deoxyribonucleotides×100)/(Length of Alignment−Total Number of Gaps in Alignment)

Signal peptide: The precursor peptides typically consist of an N-terminal leader and a C-terminal core peptide. A signal peptide governing subcellular localization may be attached to the N-terminus of the leader peptide. In eukaryotes, the signal peptide of a nascent precursor protein (pre-protein) directs the ribosome to the rough endoplasmic reticulum (ER) membrane and initiates the transport of the growing peptide chain across it. In one embodiment of the present invention, the signal peptide is encoded by a third polynucleotide, the third polynucleotide being operably linked in translational fusion to the second polynucleotide encoding a leader peptide upstream of the second polynucleotide; and the signal peptide having a sequence identity of at least 60% e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO: 4 (MRLTLLSGVAGVLCAGQLTAA), SEQ ID NO: 41 (MRLSTSSLFLSVSLLGKLALG) or SEQ ID NO: 52 (MGVSAVLLPLYLLSGVTFGLA). In a preferred embodiment the signal peptide comprises, essentially consists of, or consists of SEQ ID NO: 4, SEQ ID NO: 41 or SEQ ID NO: 52.

Depending on the terminology, the signal peptide may include a leader peptide and thereby be described as elongated signal peptide. Therefore, in one embodiment the elongated signal peptide is encoded by a third polynucleotide, the third polynucleotide being operably linked in translational fusion to the first polynucleotide encoding a polypeptide of interest upstream of the first polynucleotide; and the elongated signal peptide having a sequence identity of at least 60% e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO: 6 (MRLTLLSGVAGVLCAGQLTAAFARAPVAAR), SEQ ID NO: 43 (MRLSTSSLFLSVSLLGKLALGFARAPVAAR) or SEQ ID NO: 45 (MGVSAVLLPLYLLSGVTFGLAFARAPVAAR).

Translational fusion: The first and second polynucleotide are operably linked in translational fusion. In the context of the present invention, the term “operably linked in translation fusion” means that the leader peptide encoded by the second polynucleotide and the polypeptide of interest encoded by the first polynucleotide are encoded in frame and translated together as a single polypeptide. Following translation, the leader peptide is removed to provide the mature polypeptide of interest. Additionally or alternatively, a third polynucleotide encoding a signal peptide is operably linked in translational fusion to the second polynucleotide upstream of the second polynucleotide, said second polynucleotide being operably linked in translational fusion to the first polynucleotide. Following translation, the signal peptide and leader peptide are removed to provide the mature polypeptide of interest. Preferably, the mature polypeptide of interest is secreted.

Variant: The term “variant” means a polypeptide having glucoamylase activity comprising a man-made mutation, i.e., a substitution, insertion, and/or deletion (e.g., truncation), at one or more (e.g., several) positions to improve the expression and/or thermostability. A substitution means replacement of the amino acid occupying a position with a different amino acid; a deletion means removal of the amino acid occupying a position; and an insertion means adding an amino acid adjacent to and immediately following the amino acid occupying a position. Additionally or alternatively, the term “variant” means a polypeptide having biological activity comprising one or more of a leader peptide, a signal peptide and an elongated signal peptide.

Wild-type: The term “wild-type” in reference to an amino acid sequence or nucleic acid sequence means that the amino acid sequence or nucleic acid sequence is a native or naturally-occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild- type sequence).

### DETAILED DESCRIPTION OF THE INVENTION

Host Cells The present invention also related to recombinant host cells, comprising a polynucleotide of the present invention operably linked to one or more control sequences that direct the production of a polypeptide of interest. A construct or vector comprising a polynucleotide is introduced into a host cell so that the construct or vector is maintained as a chromosomal integrant or as a self-replicating extra-chromosomal vector as described earlier. The choice of the host cell will to a large extent depend upon the gene encoding the polypeptide and its source.

In some embodiments, the polypeptide is heterologous to the recombinant host cell.

In some embodiments, at least one of the one or more control sequences is heterologous to the polynucleotide encoding the polypeptide of interest, the signal peptide, and/or the leader peptide.

In some embodiments, the recombinant host cell comprises at least two copies, e.g., three, four, or five copies of the polynucleotide of the present invention.

The host cell may be any microbial cell useful in the recombinant production of a polypeptide of interest, e.g. a fungal host cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolismis obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viridecell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

In a first aspect, the invention relates to a fungal host cell comprising in its genome:
a first polynucleotide encoding a polypeptide of interest; and
a second polynucleotide operably linked in translational fusion to the first polynucleotide upstream of the first polynucleotide, said second polynucleotide encoding a leader peptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO: 2 (FARAPVAAR). As presented throughout the examples, host cells with said leader peptide operably linked to a polypeptide of interest have surprisingly shown increased expression, product yield and/or product activity.

In an embodiment of the first aspect, the leader peptide comprises, consists essentially of, or consists of SEQ ID NO: 2.

In one embodiment, the leader peptide is synthetic.

In a preferred embodiment, the leader peptide is heterologous to the polypeptide of interest.

In another preferred embodiment, the leader peptide is heterologous to the signal peptide. In another preferred embodiment, the leader peptide is heterologous to the signal peptide and to the polypeptide of interest.

In another embodiment, the second polynucleotide encoding the leader peptide of SEQ ID NO: 2 comprises one or more mutations, preferably nucleotide substitutions, nucleotide deletions or nucleotide insertions. Said mutation(s) leading to a variant of the leader peptide of SEQ ID NO: 2, such as a variant comprising (i) one or more additional amino acids compared to SEQ ID NO:2, (ii) at least one amino acid less compared to SEQ ID NO: 2, e.g. a total of 3 to 8 amino acids, (iii) or an amino acid substitution of at least one amino acid of SEQ ID NO: 2, such as a substitution of the amino acid at a position corresponding to position 1, 2, 3, 4, 5, 6, 7, 8 or 9 of SEQ ID NO: 2.

In a further embodiment, the host cell comprises in its genome a third polynucleotide encoding a signal peptide, wherein the third polynucleotide is operably linked in translational fusion to the second polynucleotide upstream of the second polynucleotide; and wherein the polypeptide of interest is secreted.

In another embodiment, the third polynucleotide encodes a signal peptide having a sequence identity of at least 60% e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO: 4 (MRLTLLSGVAGVLCAGQLTAA), SEQ ID NO: 41 (MRLSTSSLFLSVSLLGKLALG) or SEQ ID NO: 52 (MGVSAVLLPLYLLSGVTFGLA). In a preferred embodiment, the third polynucleotide consists of, essentially consists of, or comprises SEQ ID NO: 4, SEQ ID NO: 41 or SEQ ID NO: 52.

In another embodiment, the third polynucleotide encoding the signal peptide comprises one or more mutations, preferably nucleotide substitutions, nucleotide deletions or nucleotide insertions. Said mutation(s) leading to a variant of the signal peptide of SEQ ID NO: 4, SEQ ID NO: 41 or SEQ ID NO: 52, such as a variant comprising (i) one or more additional amino acids compared to SEQ ID NO: 4, SEQ ID NO: 41 or SEQ ID NO: 52, (ii) at least one amino acid less compared to SEQ ID NO: 4, SEQ ID NO: 41 or SEQ ID NO: 52, e.g. a total of 10 to 20 amino acids, (iii) or an amino acid substitution of at least one amino acid of SEQ ID NO: 4, SEQ ID NO:

41 or SEQ ID NO: 52, such as a substitution of the amino acid at a position corresponding to position 1, 2, 3, 4, 5, 6, 7, 8, 9, 10, 11, 12, 13, 14, 15, 16, 17, 18, 19, 20, or 21 of SEQ ID NO: 4, SEQ ID NO: 41 or SEQ ID NO: 52.

In another embodiment, the fungal host cell is a yeast host cell, preferably the yeast host cell is selected from the group consisting of Candida, Hansenula, Kluyveromyces, Pichia(Komagataella), Saccharomyces, Schizosaccharomyces, and Yarrowia cell; more preferably the yeast host cell is selected from the group consisting of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica cell, most preferably Pichia pastoris (Komagataella phaffii).

In one embodiment the fungal host cell is a filamentous fungal host cell; preferably the filamentous fungal host cell is selected from the group consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma cell; more preferably the filamentous fungal host cell is selected from the group consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride cell; even more preferably the filamentous host cell is selected from the group consisting of Aspergillus oryzae, Fusarium venenatum, and Trichoderma reesei cell; most preferably the filamentous fungal host cell is an Aspergillus nigercell. In another preferred embodiment the filamentous fungal host cell is an Aspergillus oryzaecell. In yet another preferred embodiment the filamentous fungal host cell is a Trichoderma reeseicell.

In another preferred embodiment, the polypeptide of interest comprises an enzyme; preferably the enzyme is selected from the group consisting of hydrolase, isomerase, ligase, lyase, oxidoreductase, or transferase; more preferably an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase.

In a preferred embodiment, the polypeptide of interest is a glycoprotein, preferably an alpha-glucosidase; more preferably an 1,4-alpha-glucosidase; most preferably a glucoamylase, such as a glucoamylase having a sequence identity of at least 60% to SEQ ID NO: 15, SEQ ID NO: 16 , SEQ ID NO: 17 or SEQ ID NO: 18.

In one embodiment, the fungal host cell is comprising a polypeptide, said polypeptide comprising a leader peptide operably linked in translational fusion to a polypeptide of interest, wherein
(i) the leader peptide has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO: 2 (FARAPVAAR); OR
(ii) the leader peptide comprises, consists essentially of, or consists of SEQ ID NO: 2.

Additionally or alternatively, the polypeptide also comprises a signal peptide upstream of the leader peptide having a sequence identity of at least 60% e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO: 4 (MRLTLLSGVAGVLCAGQLTAA), SEQ ID NO: 41 (MRLSTSSLFLSVSLLGKLALG) or SEQ ID NO: 52 (MGVSAVLLPLYLLSGVTFGLA).

In one embodiment, the signal peptide upstream of the leader peptide comprises, essentially consists of, or consists of SEQ ID NO: 4, SEQ ID NO: 41 or SEQ ID NO: 52.

### Methods of Production

In a second aspect, the present invention relates to a method for producing a polypeptide of interest, the method comprising:
(i) providing a fungal host cell according to the first aspect,
(ii) cultivating said fungal host cell under conditions conducive for expression of the polypeptide of interest; and, optionally
(iii) recovering the polypeptide of interest.

The host cells are cultivated in a nutrient medium suitable for production of the polypeptide using methods known in the art and as described in the Examples below. For example, the cells may be cultivated by shake flask (SF) cultivation, or small-scale or large-scale fermentation (including continuous, batch, fed-batch, or solid-state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates. As shown throughout the examples, the inventors have surprisingly found that the increased expression, activity and/or yield of the polypeptide of interest can be achieved by using different cultivation media during the production process.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide

The polypeptide may be recovered using methods known in the art. For example, the polypeptide may be recovered from the fermentation medium by conventional procedures including, but not limited to, collection, centrifugation, filtration, extraction, spray-drying, evaporation, or precipitation. In one aspect, a whole fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

### Polypeptides Having Glucoamylase Activity

In some embodiments, the present invention relates to isolated or purified polypeptides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 15, SEQ ID NO: 16, SEQ ID NO: 17 or SEQ ID NO: 18, which have glucoamylase activity. In one aspect, the polypeptides differ by up to 10 amino acids, e.g., 1, 2, 3, 4, 5, 6, 7, 8, 9, or 10, from the mature polypeptide of SEQ ID NO: 15, SEQ ID NO: 16, SEQ ID NO: 17, SEQ ID NO: 18, SEQ ID NO: 47, SEQ ID NO: 49, or SEQ ID NO: 51.

The polypeptide preferably comprises, consists essentially of, or consists of the amino acid sequence of SEQ ID NO: 15, SEQ ID NO: 16, SEQ ID NO: 17, SEQ ID NO: 18, SEQ ID NO: 47, SEQ ID NO: 49, or SEQ ID NO: 51., or the mature polypeptide thereof; or is a fragment thereof having glucoamylase activity. In one aspect, the mature polypeptide is SEQ ID NO: 15. In another aspect the mature polypeptide is SEQ ID NO: 16. In another aspect the mature polypeptide is SEQ ID NO: 17. In yet another aspect the mature polypeptide is SEQ ID NO: 18.

In some embodiments, the present invention relates to isolated or purified polypeptides having glucoamylase activity encoded by polynucleotides that hybridize under medium stringency conditions, medium-high stringency conditions, high stringency conditions, or very high stringency conditions with the full-length complement of the mature polypeptide coding sequence of SEQ ID NO: 7, 9, 11, 13 or the cDNA thereof (Sambrook et al., 1989, Molecular Cloning, A Laboratory Manual, 2d edition, Cold Spring Harbor, New York).

The polynucleotide of SEQ ID NO: 7, SEQ ID NO: 9, SEQ ID NO: 11, SEQ ID NO: 13, SEQ ID NO: 46, SEQ ID NO: 48, or SEQ ID NO: 50or a subsequence thereof, as well as the mature polypeptide of SEQ ID NO: 8, 10, 12, 14, 15, 16, 17, 18 or a fragment thereof, may be used to design nucleic acid probes to identify and clone DNA encoding polypeptides having glucoamylase activity from strains of different genera or species according to methods well known in the art. Such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide having glucoamylase activity. Genomic or other DNA from such other strains may be separated by agarose or polyacrylamide gel electrophoresis, or other separation techniques. DNA from the libraries or the separated DNA may be transferred to and immobilized on nitrocellulose or another suitable carrier material. In order to identify a clone or DNA that hybridizes with SEQ ID NO: 7, SEQ ID NO: 9, SEQ ID NO: 11, SEQ ID NO: 13, SEQ ID NO: 46, SEQ ID NO: 48, or SEQ ID NO: 50 or a subsequence thereof, the carrier material is used in a Southern blot.

For purposes of the present invention, hybridization indicates that the polynucleotides hybridize to a labeled nucleic acid probe corresponding to (i) SEQ ID NO: 7, SEQ ID NO: 9, SEQ ID NO: 11, SEQ ID NO: 13, SEQ ID NO: 46, SEQ ID NO: 48, or SEQ ID NO: 50; (ii) the mature polypeptide coding sequence of SEQ ID NO: 7, SEQ ID NO: 9, SEQ ID NO: 11, SEQ ID NO: 13,

SEQ ID NO: 46, SEQ ID NO: 48, or SEQ ID NO: 50; (iii) the full-length complement thereof; or (iv) a subsequence thereof; under medium to very high stringency conditions. Molecules to which the nucleic acid probe hybridizes under these conditions can be detected using, for example, X-ray film or any other detection means known in the art.

In some embodiments, the present invention relates to isolated polypeptides having glucoamylase activity encoded by polynucleotides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 7, SEQ ID NO: 9, SEQ ID NO: 11, SEQ ID NO: 13, SEQ ID NO: 46, SEQ ID NO: 48, or SEQ ID NO: 50.

The polynucleotide encoding the polypeptide preferably comprises, consists essentially of, or consists of nucleotides 91 to 1878 of SEQ ID NO: 7, SEQ ID NO: 9, SEQ ID NO: 11, SEQ ID NO: 13, SEQ ID NO: 46, SEQ ID NO: 48, or SEQ ID NO: 50.

In some embodiments, the present invention relates to a polypeptide derived from a mature polypeptide of SEQ ID NO: 10 or 16 by substitution, deletion or addition of one or several amino acids in the mature polypeptide of SEQ ID NO: 10 or 16. In some embodiments, the present invention relates to variants of the mature polypeptide of SEQ ID NO: 10 or 16 comprising a substitution, deletion, and/or insertion at one or more (e.g., several) positions. In one aspect, the number of amino acid substitutions, deletions and/or insertions introduced into the mature polypeptide of SEQ ID NO: 10 or 16 is up to 10, e.g., 1, 2, 3, 4, 5, 6, 7, 8, 9, or 10. In an embodiment, the polypeptide has an N-terminal extension and/or C-terminal extension of 1-10 amino acids, e.g., 1, 2, 3, 4, 5, 6, 7, 8, 9, or 10 amino acids. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

In some embodiments, the present invention relates to a polypeptide derived from a mature polypeptide of SEQ ID NO: 12 or 17 by substitution, deletion or addition of one or several amino acids in the mature polypeptide of SEQ ID NO: 12 or 17. In some embodiments, the present invention relates to variants of the mature polypeptide of SEQ ID NO: 12 or 17 comprising a substitution, deletion, and/or insertion at one or more (e.g., several) positions. In one aspect, the number of amino acid substitutions, deletions and/or insertions introduced into the mature polypeptide of SEQ ID NO: 12 or 17 is up to 10, e.g., 1, 2, 3, 4, 5, 6, 7, 8, 9, or 10. In an embodiment, the polypeptide has an N-terminal extension and/or C-terminal extension of 1-10 amino acids, e.g., 1, 2, 3, 4, 5, 6, 7, 8, 9, or 10 amino acids. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

In some embodiments, the present invention relates to a polypeptide derived from a mature polypeptide of SEQ ID NO: 14 or 18 by substitution, deletion or addition of one or several amino acids in the mature polypeptide of SEQ ID NO: 14 or 18. In some embodiments, the present invention relates to variants of the mature polypeptide of SEQ ID NO: 14 or 18 comprising a substitution, deletion, and/or insertion at one or more (e.g., several) positions. In one aspect, the number of amino acid substitutions, deletions and/or insertions introduced into the mature polypeptide of SEQ ID NO: 14 or 18 is up to 10, e.g., 1, 2, 3, 4, 5, 6, 7, 8, 9, or 10. In an embodiment, the polypeptide has an N-terminal extension and/or C-terminal extension of 1-10 amino acids, e.g., 1, 2, 3, 4, 5, 6, 7, 8, 9, or 10 amino acids. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

In some embodiments, the present invention relates to a polypeptide derived from a mature polypeptide of SEQ ID NO: 16 by substitution of one or several amino acids in the mature polypeptide of SEQ ID NO: 16. In some embodiments, the present invention relates to variants of the mature polypeptide of SEQ ID NO: 16 comprising a substitution, deletion, and/or insertion at one or more (e.g., several) positions. The number of amino acid substitutions, deletions and/or insertions introduced into the mature polypeptide of SEQ ID NO: 16 is up to 20, e.g., 1, 2, 3, 4, 5, 6, 7, 8, 9, 10, 11, 12, 13, 14, 15, 16, 17, 18, 19 or 20. In some embodiments the substitutions are selected from a substitution at a position corresponding to position 6, 7, 31, 34, 103, 132, 445, 447, 481, 566, 568, 594, or 595 of SEQ ID NO: 16. In some embodiments the substitutions are selected from a substitution at a position corresponding to position 6, 7, 31, 34, 103, 132, 445, 447, 481, 566, 568, 594, or 595 of SEQ ID NO: 16, wherein the substitutions are one or more of G6S, G7T, R31F, K34Y, S103N, A132P, D445N, V447S, S481P, D566T, T568V, Q594R, or F595S. In one embodiment the variant polypeptide of SEQ ID NO: 16 is the polypeptide comprising, essentially consisting of, or consisting of SEQ ID NO: 17.

In some embodiments the substitutions are selected from a substitution at a position corresponding to position 6, 7, 31, 34, 50, 103, 132, 445, 447, 481, 484, 501, 539, 566, 568, 594 or 595 of SEQ ID NO: 16. In some embodiments the substitutions are selected from a substitution at a position corresponding to 6, 7, 31, 34, 50, 103, 132, 445, 447, 481, 484, 501, 539, 566, 568, 594 or 595 of SEQ ID NO: 16, wherein the substitutions are one or more of G6S, G7T, R31F, K34Y, E50R, S103N, A132P, D445N, V447S, S481P, T484P, E501A, N539P, D566T, T568V, Q594R, or F595. In one embodiment the variant polypeptide of SEQ ID NO: 16 is the polypeptide comprising, essentially consisting of, or consisting of SEQ ID NO: 18.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are tested for glucoamylase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide. With regards to thermostability and/or enzymatic activity, essential amino acids in the sequence of amino acids 1 to 595 of SEQ ID NO: 16 are located at positions 6, 7, 31, 34, 50, 103, 132, 445, 447, 481, 484, 501,539, 566, 568, 594, or 595.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

In some embodiments, the polypeptide is a fragment containing at least 100 amino acid residues of the mature polypeptide of SEQ ID NO: 15, SEQ ID NO: 16, SEQ ID NO: 17, SEQ ID NO: 18, SEQ ID NO: 47, SEQ ID NO: 49, or SEQ ID NO: 51, at least 300 amino acid residues of the mature polypeptide of SEQ ID NO: 15, SEQ ID NO: 16, SEQ ID NO: 17, SEQ ID NO: 18, SEQ ID NO: 47, SEQ ID NO: 49, or SEQ ID NO: 51, or at least 400 amino acid residues of the mature polypeptide of SEQ ID NO: 15, SEQ ID NO: 16, SEQ ID NO: 17, SEQ ID NO: 18, SEQ ID NO: 47, SEQ ID NO: 49, or SEQ ID NO: 51.

The polypeptide may be a hybrid polypeptide or a fusion polypeptide.

The polypeptides of the present invention have improved thermostability and improved expression in fungal host cells.

### Polynucleotides

The present invention also relates to isolated polynucleotides encoding a polypeptide of interest, a signal peptide, an elongated signal peptide or a leader peptide of the present invention, as described herein.

The techniques used to isolate or clone a polynucleotide are known in the art and include isolation from genomic DNA or cDNA, or a combination thereof. The cloning of the polynucleotides from genomic DNA can be affected, e.g., by using the polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Aspergillus niger, Penicillum oxalicum, Rasamsonia emersonii, or a related organism and thus, for example, may be a species variant of the polypeptide encoding region of the polynucleotide.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of the polynucleotide presented as the mature polypeptide coding sequence of SEQ ID NO: 1, 3, 5, 9, e.g., a subsequence thereof, and/or by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

### Nucleic Acid Constructs

The present invention also relates to nucleic acid constructs comprising a polynucleotide of the present invention, wherein the polynucleotide is operably linked to one or more control sequences that direct the expression of the coding sequence in a suitable host cell under conditions compatible with the control sequences.

In a third aspect, the present invention relates to a nucleic acid construct comprising a first polynucleotide encoding a polypeptide of interest, and a second polynucleotide operably linked to the first polynucleotide encoding a leader peptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO: 2 (FARAPVAAR).

In one embodiment of the third aspect, the leader peptide comprises, consists essentially of, or consists of SEQ ID NO:2.

In one embodiment the, leader peptide is synthetic.

In a preferred embodiment, the leader peptide is heterologous to the polypeptide of interest.

In another preferred embodiment, the leader peptide is heterologous to the signal peptide. In another preferred embodiment, the leader peptide is heterologous to the signal peptide and to the polypeptide of interest.

In another embodiment, the second polynucleotide encoding the leader peptide of SEQ ID NO: 2 comprises one or more mutations, preferably nucleotide substitutions, nucleotide deletions or nucleotide insertions. Said mutation(s) leading to a variant of the leader peptide of SEQ ID NO: 2, such as a variant comprising (i) one or more additional amino acids compared to SEQ ID NO: 2, (ii) at least one amino acid less compared to SEQ ID NO: 2, e.g. a total of 4 to 8 amino acids, (iii) or an amino acid substitution of at least one amino acid of SEQ ID NO: 2, such as a substitution of the amino acid at a position corresponding to position 1, 2, 3, 4, 5, 6, 7, 8, or 9 of SEQ ID NO: 2.

In a further embodiment, the second polynucleotide is operably linked to one or more control sequences that direct the production of the polypeptide in an expression host.

In another embodiment, the nucleic acid construct additionally or alternatively comprises a third polynucleotide encoding a signal peptide,
wherein the third polynucleotide is operably linked in translational fusion to the second polynucleotide upstream of the second polynucleotide; and
the signal peptide having a sequence identity of at least 60% e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO: 4 (MRLTLLSGVAGVLCAGQLTAA), SEQ ID NO: 41 or SEQ ID NO: 52. In a preferred embodiment, the signal peptide consists of, essentially consists of, or comprises SEQ ID NO: 4, SEQ ID NO: 41 or SEQ ID NO: 52. In yet another preferred embodiment, the third polynucleotide is operably linked to one or more control sequences that direct the production of the polypeptide in an expression host.

In another embodiment, the third polynucleotide encoding the signal peptide comprises one or more mutations, preferably nucleotide substitutions, nucleotide deletions or nucleotide insertions. Said mutation(s) leading to a variant of the signal peptide of SEQ ID NO: 4, SEQ ID NO: 41 or SEQ ID NO: 52, such as a variant comprising (i) one or more additional amino acids compared to SEQ ID NO: 4, SEQ ID NO: 41 or SEQ ID NO: 52, (ii) at least one amino acid less compared to SEQ ID NO: 4, SEQ ID NO: 41 or SEQ ID NO: 52, e.g. a total of 10 to 20 amino acids, (iii) or an amino acid substitution of at least one amino acid of SEQ ID NO: 4, SEQ ID NO: 41 or SEQ ID NO: 52, such as a substitution of the amino acid at a position corresponding to position 1, 2, 3, 4, 5, 6, 7, 8, 9, 10, 11, 12, 13, 14, 15, 16, 17, 18, 19, 20, or 21 of SEQ ID NO: 4, SEQ ID NO: 41 or SEQ ID NO: 52.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide with the leader peptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the polynucleotide of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the polynucleotide of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillusneutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus nigerglucoamylase, Aspergillus niger alpha-glucosidase, Aspergillus oryzae TAKA amylase, Fusarium oxysporum trypsin-like protease, Trichoderma reesei beta-glucosidase, Trichoderma reeseicellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, J. Bacteriol. 177: 3465-3471).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide, such as the signal peptide of SEQ ID NO: 4, SEQ ID NO: 41 or SEQ ID NO: 52, or the elongated signal peptide of SEQ ID NO: 6, SEQ ID NO 43 or SEQ ID NO: 45. Alternatively, the 5′-end of the coding sequence may contain a signal peptide coding sequence that is heterologous to the coding sequence. A heterologous signal peptide coding sequence may be required where the coding sequence does not naturally contain a signal peptide coding sequence. Alternatively, a heterologous signal peptide coding sequence may simply replace the natural signal peptide coding sequence to enhance secretion of the polypeptide. However, any signal peptide coding sequence that directs the expressed polypeptide into the secretory pathway of a host cell may be used. In a preferred embodiment, the signal peptide comprises, essentially consists of, or consists of SEQ ID NO: 4, SEQ ID NO: 41 or SEQ ID NO: 52. In another preferred embodiment, the signal peptide comprises, essentially consists of, or consists of SEQ ID NO: 6, SEQ ID NO: 43 or SEQ ID NO: 45. Alternatively, the signal peptide has a sequence identity of at least 60% e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO: 4, SEQ ID NO: 6, SEQ ID NO: 41, SEQ ID NO: 43, SEQ ID NO: 45 or SEQ ID NO: 52.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiol. Rev. 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence. In a preferred embodiment, the propeptide is a leader peptide with SEQ ID NO: 2. Alternatively, the propeptide is a leader peptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO: 2.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reeseicellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide of interest at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

In a fourth aspect, the present invention relates to an expression vector comprising a nucleic acid construct according to the third aspect.

The recombinant expression vector may be any vector (e.g., a plasmid or virus) that can be conveniently subjected to recombinant DNA procedures and can bring about expression of the polynucleotide along with the leader peptide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is a hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMß1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of the polypeptide of interest. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Signal Peptide and Leader Peptide

The present invention also relates to an isolated polynucleotide encoding an signal peptide comprising or consisting of amino acids 1 to 21 of SEQ ID NO: 4, amino acids 1 to 21 of SEQ ID NO: 6 or amino acids 1 to 21 of SEQ ID NO: 10, SEQ ID NO: 41 or SEQ ID NO: 52. The present invention also relates to an isolated polynucleotide encoding a synthetic leader peptide comprising or consisting of amino acids 1 to 9 of SEQ ID NO: 2, amino acids 22 to 30 of SEQ ID NO: 6, amino acids 22 to 30 of SEQ ID NO: 10, amino acids 22 to 30 of SEQ ID NO: 43 or amino acids 22 to 30 of SEQ ID NO: 45. In one embodiment, the polynucleotide encoding the leader peptide of SEQ ID NO: 2 comprises one or more mutations, preferably nucleotide substitutions, nucleotide deletions or nucleotide insertions. Said mutation(s) leading to a variant of the signal peptide of SEQ ID NO: 2, such as a variant comprising (i) one or more additional amino acids compared to SEQ ID NO: 2, (ii) at least one amino acid less compared to SEQ ID NO: 2, e.g. a total of 4 to 8 amino acids, (iii) or an amino acid substitution of at least one amino acid of SEQ ID NO: 2, such as a substitution of the amino acid at a position corresponding to position 1, 2, 3, 4, 5, 6, 7, 8, or 9 of SEQ ID NO: 2.

In another embodiment the polynucleotide is encoding a leader peptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO: 2 (FARAPVAAR).

The present invention also relates to an isolated polynucleotide encoding a signal peptide and a leader peptide comprising or consisting of amino acids 1 to 30 of SEQ ID NO: 6, amino acids 1 to 30 of SEQ ID NO: 10, amino acids 1 to 30 of SEQ ID NO: 43 or amino acids 1 to 30 of SEQ ID NO: 45. Preferably, the polynucleotide is encoding a signal peptide and a leader peptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO: 6, SEQ ID NO: 43 or SEQ ID NO: 45.

The polynucleotides may further comprise a gene encoding a protein, which is operably linked to the signal peptide and/or leader peptide, such as a glucoamylase. The protein is preferably heterologous to the signal peptide and/or leader peptide. In one aspect, the polynucleotide encoding the signal peptide is nucleotides 1 to 63 of SEQ ID NO: 3, SEQ ID 42, SEQ ID NO: 44, SEQ ID NO: 46, SEQ ID NO: 48 or SEQ ID NO: 50. In another aspect, the polynucleotide encoding the leader peptide is nucleotides 1 to 27 of SEQ ID NO: 1. In another aspect, the polynucleotide encoding the signal peptide and the leader peptide is nucleotides 1 to 90 of SEQ ID NO: 5, SEQ ID 42, SEQ ID NO: 44, SEQ ID NO: 46, SEQ ID NO: 48 or SEQ ID NO: 50.

The present invention also relates to nucleic acid constructs, expression vectors and recombinant host cells comprising such polynucleotides, in particular fungal host cells.

The present invention also relates to methods of producing a protein, comprising (a) cultivating a recombinant host cell comprising such polynucleotide; and optionally (b) recovering the protein.

The protein may be native or heterologous to a host cell. The term “protein” is not meant herein to refer to a specific length of the encoded product and, therefore, encompasses peptides, oligopeptides, and polypeptides. The term “protein” also encompasses two or more polypeptides combined to form the encoded product. The proteins also include hybrid polypeptides and fused polypeptides.

Preferably, the protein is a hormone, enzyme, receptor or portion thereof, antibody or portion thereof, or reporter. For example, the protein may be a hydrolase, isomerase, ligase, lyase, oxidoreductase, or transferase, e.g., an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase. Preferably the protein is a glucoamylase.

The gene may be obtained from any prokaryotic, eukaryotic, or other source.

The present invention is further described by the following examples that should not be construed as limiting the scope of the invention.

### EXAMPLES

### Materials and Methods

Unless otherwise stated, DNA manipulations and transformations were performed using standard methods of molecular biology as described in Sambrook et al. (1989) Molecular cloning: A laboratory manual, Cold Spring Harbor lab., Cold Spring Harbor, NY; Ausubel, F. M. et al. (eds.) “Current protocols in Molecular Biology”, John Wiley and Sons, 1995; Harwood, C. R., and Cut-ting, S. M. (eds.) “Molecular Biological Methods for Bacillus”. John Wiley and Sons, 1990.

Purchased Material (E. coli and kits)

Amplified plasmids are recovered with Qiagen Plasmid Kit (Qiagen). Ligation is done with either Rapid DNA Dephos & Ligation Kit (Roche) or In-Fusion kit (Clontech Laboratories, Inc.) according to the manufactory instructions. Polymerase Chain Reaction (PCR) is carried out with KOD-Plus system (TOYOBO). Fungal spore-PCR was conducted by using Phire® Plant Direct PCR Kit (New England Biolabs). QIAquick™ Gel Extraction Kit (Qiagen) is used for the purification of PCR fragments and extraction of DNA fragment from agarose gel.

### Enzymes

Enzymes for DNA manipulations (e.g. restriction endonucleases, ligases etc.) are obtainable from New England Biolabs, Inc. and were used according to the manufacturer's instructions.

### Plasmids

The sequence for the amyloglucosidase from Penicillium oxalicum is described in WO2011/127802 (SEQ ID NO: 2). pHUda1511 was AnPav498 vector. The sequence for the amylase from Rhizomucor pusillus is described in EP2527448-A1 (SEQ ID 84). The pJaL 1470 is described in WO2015144936A1.

### Microbial Strains

The expression host strain Aspergillus niger M1396 and M1412 (pyrG-phenotype/ uridine auxotrophy) was isolated by Novozymes and is a derivative of Aspergillus niger NN049184 which was isolated from soil described in example 14 in WO2012/160093. C2446, C2661, C5502, C5503 and C5553 are strains which can produce the glucoamylase (1,4-alpha-D-glucan glucohydrolase, EC 3.2.1.3) from Penicillium oxalicum.

The expression host strain Aspergillus niger C2446, C2661, C5502, C5503 and C5553 (pyrG-pheno-type/ uridine auxotrophy) were isolated by Novozymes and were derivatives of Aspergillus niger NN049184 which was isolated from soil as described in example 14 in WO2012/160093. C2578 and M1328 (pyrG-phenotype of C2578) are strains which can produce the glucoamylase from Penicillium oxalicum.

### Medium

COVE trace metals solution was composed of 0.04 g of NaB4O7·10H2O, 0.4 g of CuSO4·5H2O, 1.2 g of FeSO4·7H2O, 0.7 g of MnSO4·H2O, 0.8 g of Na2MoO2·2H20, 10 g of ZnSO4·7H2O, and deionized water to 1 liter.

50× COVE salts solution was composed of 26 g of KCl, 26 g of MgSO4·7H2O, 76 g of KH2PO4, 50 ml of COVE trace metals solution, and deionized water to 1 liter.

COVE medium was composed of 342.3 g of sucrose, 20 ml of 50× COVE salts solution, 10 ml of 1 M acetamide, 10 ml of 1.5 M CsCl2, 25 g of Noble agar, and deionized water to 1 liter.

COVE-N-Gly plates were composed of 218 g of sorbitol, 10 g of glycerol, 2.02 g of KNO3, 50 ml of COVE salts solution, 25 g of Noble agar, and deionized water to 1 liter.

COVE-N (tf) was composed of 342.3 g of sucrose, 3 g of NaNO3, 20 ml of COVE salts solution, 30 g of Noble agar, and deionized water to 1 liter.

COVE-N top agarose was composed of 342.3 g of sucrose, 3 g of NaNO3, 20 ml of COVE salts solution, 10 g of low melt agarose, and deionized water to 1 liter.

COVE-N was composed of 30 g of sucrose, 3 g of NaNO3, 20 ml of COVE salts solution, 30 g of Noble agar, and deionized water to 1 liter.

STC buffer was composed of 0.8 M sorbitol, 25 mM Tris pH 8, and 25 mM CaCl2.

STPC buffer was composed of 40% PEG 4000 in STC buffer.

LB medium was composed of 10 g of tryptone, 5 g of yeast extract, 5 g of sodium chloride, and deionized water to 1 liter.

LB plus ampicillin plates were composed of 10 g of tryptone, 5 g of yeast extract, 5 g of sodium chloride, 15 g of Bacto agar, ampicilin at 100 μg per ml, and deionized water to 1 liter.

YPG medium was composed of 10 g of yeast extract, 20 g of Bacto peptone, 20 g of glucose, and deionized water to 1 liter.

SOC medium was composed of 20 g of tryptone, 5 g of yeast extract, 0.5 g of NaCl, 10 ml of 250 mM KCl, and deionized water to 1 liter.

TAE buffer was composed of 4.84 g of Tris Base, 1.14 ml of Glacial acetic acid, 2 ml of 0.5 M EDTA pH 8.0, and deionized water to 1 liter.
MSS is composed of 70 g Sucrose, 100 g Soybean powder (pH 6.0), water to 1 liter.
MU-1 is composed 260 g of Maltodextrin, 3 g of MgSO4·7H2O, 5 g of KH2PO4, 6 g of K2SO4, amyloglycosidase trace metal solution 0.5 ml and urea 2 g (pH 4.5), water to 1 li-ter.
MU-1 glu is composed 260 g of glucose, 3 g of MgSO4·7H2O, 5 g of KH2PO4, 6 g of K2SO4, amyloglycosidase trace metal solution 0.5 ml and urea 2 g (pH 4.5), water to 1 li-ter. CDM2 medium (pH 6.5) was composed of 30 g of Sucrose, 3 g of NaNO3, 1 g of K2HPO4, 0.5 g of MgSO4 7H2O, 0.5 g of KCl, 0.01 g of FeSO4 7H2O, 20 g of Maltose H2O, 20 g of Agar, BA-10, and deionized water to 1 liter.

Pullulan medium was composed of 0.2 g of Pullulan, 1 g of NaNO3, 1 g of Agar, BA-10, 0.1 g of Sodium azide, 5 mL of 1 M Acetate buffer (pH4.3) and deionized water to 100 ml.

Transformation of Aspergillus niger

Transformation of Aspergillus species can be achieved using the general methods for yeast transformation. The preferred procedure for the invention is described below.

Aspergillus niger host strain was inoculated to 100 ml of YPG medium supplemented with 10 mM uridine and incubated for 16 hrs at 32° C. at 80 rpm. Pellets were collected and washed with 0.6 M KCl, and resuspended 20 ml 0.6 M KCl containing a commercial beta-glucanase product (GLUCANEX™, Novozymes A/S, Bagsværd, Denmark) at a final concentration of 20 mg per ml. The suspension was incubated at 32° C. at 80 rpm until protoplasts were formed, and then washed twice with STC buffer. The protoplasts were counted with a hematometer and resuspended and adjusted in an 8:2:0.1 solution of STC:STPC:DMSO to a final concentration of 2.5×107 protoplasts/ml. Approximately 4 μg of plasmid DNA was added to 100 μl of the protoplast suspension, mixed gently, and incubated on ice for 30 minutes. One ml of SPTC was added and the protoplast suspension was incubated for 20 minutes at 37° C. After the addition of 10 ml of 50° C. Cove or Cove-N top agarose, the reaction was poured onto Cove or Cove-N (tf) agar plates and the plates were incubated at 32° C. for 5 days.

### PCR Amplifications in Examples

Polymerase Chain Reaction (PCR) was carried out with PrimeSTAR Max DNA polymerase [TaKaRa].

| Component | Volume | Final Concentration |
|---|---|---|
| 2xPrimeSTAR Max DNA polymerase mix- | 25 μl | 1x |
| 10 pmol/μl Primer #1 | 1.5 μl | 0.3 μM |
| 10 pmol/μl Primer #2 | 1.5 μl | 0.3 μM |
| Template DNA | | |
| Genomic DNA Plasmid DNA | X μl | |
| 10-200 ng/50 μl | | |
| 1-50 ng/50 μl | | |
| PCR grade water | Y μl | |
| Total reaction volume | 50 μl | |
| 3-step cycle: | | |
| Pre-denaturation: 96° C., 2 min. | | |
| Denaturation: 96° C., 15 sec. | | |
| Annealing: Tm-[5-10] ° C.*, 30 sec. | {close oversize brace} | 35 cycles |
| Extension: 72° C., 10 s./kb | | |

### Fungal Spore-PCR

Fungal spore-PCR was conducted by using Phire® Plant Direct PCR Kit (New England Biolabs). Spores from each fungal strain were picked with a 1 μl inoculating loop and suspend in 10 μl Dilution Buffer (included in the kit). PCR cocktails were set-up as seen below.

| Component | Volume | |
|---|---|---|
| Sterile diH20 (μL) | 7.1 | |
| 2x Phire ® Plant PCR Buffer (μL) | 10 | |
| template (μL) | 0.5 | |
| 10 μM 5′-primer (μL) | 1 | |
| 10 μM 3′-primer (μL) | 1 | |
| Phire ® Hot Start II Polymerase (μL) | 0.4 | |
| 3-step cycle: | | |
| Pre-denaturation: 98° C., 5 min. | | |
| Denaturation: 98° C., 5 sec. | | |
| Annealing: Tm-[5-10] ° C.*, 5 sec. | | |
| Extension: 72° C., 20 sec/kb | {close oversize brace} | 40 cycles |
| 72° C.-1 min | | |

### Shaking Flask Cultivation for Glucoamylase Production

Spores of the selected transformants were inoculated in 100 ml of MSS media and cultivated at 30° C. for 3 days. 10% of seed culture was transferred to MU-1 medium in lab-scale tanks with feeding the appropriate amounts of glucose and ammonium and cultivated at 34° C. for 7 days. The supernatant was obtained by centrifugation.

### Lab-Scale Tank Cultivation for Glucoamylase Production

Fermentation was done as fed-batch fermentation (H. Pedersen 2000, Appl Microbiol Biotechnol, 53: 272-277). Selected strains were pre-cultured in liquid media then grown mycelia were transferred to the tanks for further cultivation of enzyme production. Cultivation was done at pH 4.75 at 34° C. for 8 days with the feeding of glucose and ammonium without over-dosing which prevents enzyme production. For examples 7 to 9, cultivation was done at pH 5.1 at 34° C. for 8 days with the feeding of glucose and ammonium without over-dosing which prevents enzyme production. Culture supernatant after centrifugation was used for enzyme assay.

### Glucoamylase Activity

Glucoamylase activity was determined by RAG assay method (Relative AG assay, pNPG method). pNPG substrate was composed of 0.1 g of p-Nitrophenyl-beta-D-glycopyranoside (Nacalai Tesque), 10 ml of 1 M Acetate buffer (pH 4.3) and deionized water to 100 ml. From each diluted sample solution, 40 ul is added to well in duplicates for “Sample”. And 40 ul deionized water is added to a well for “Blank”. And 40 ul of AG standard solution is added as “Reference”. Using

Multidrop (Labsystem), 80 ul of pNPG substrate is added to each well. After 20 minutes at room temperature, the reaction is stopped by addition of 120 ul of Stop reagent (0.1M Borax solution). OD values are measured by microplate reader at 400 nm (Power Wave X) or at 405 nm (ELx808).

Calculation was conducted as follows:

$$\frac{( S - B ) \times F \times AGs}{Ss - Bs} = RAG / ml$$

$$S = Sample value F = dilution factor$$

$$B = Blank value AGs = AG / ml of the AG standard .$$

$$Ss = Value of AG standard$$

$$Bs = Blank of AG standard$$

$$RAG = relative amyloglucosidase unit$$

### Example 1: Construction of JPO001, JPO002 and JPO003

Glucoamylase variants JPO001, JPO002 and JPO003 were constructed as follows.

The expression vectors were constructed using inverse PCR, which means amplification of entire plasmid DNA sequences by inversely directed primers, were carried out with appropriate template plasmid DNA (e.g. plasmid DNA containing AnPav498 gene) by the following conditions. The resultant PCR fragments were purified by QIAquick Gel extraction kit [QIAGEN], and then introduced into Escherichia coli ECOS Competent E. coli DH5α [NIPPON GENE CO., LTD.]. The plasmid DNAs were extracted from E. coli transformants by MagExtractor plasmid extraction kit [TOYOBO], and then introduced into A. niger competent cells (host: C2446, C2661, C5502 and C5503).

Sequences of signal peptides and Leader peptides (*Bold character is N-terminal end of mature polypeptide of interest):

according to SEQ ID NO: 8
MRLTLLSGVAGVLCAGQLTAA R (AnPav498)
according to SEQ ID NO: 6
MRLTLLSGVAGVLCAGQLTAAFARAPVAAR A (JPO001)
according to SEQ ID NO: 25
MRLTLLSGVAGVLCAGKRTGL A (JPO002)
according to SEQ ID NO: 26
MRLTLLSGVAGVLCAGQLTAAAK R (JPO003)

TABLE 1
Primers

| SEQ ID NO: | Primer name |
|---|---|
| 19 | JPO001_F 42 |
| 20 | JPO001 R 42 |
| 21 | JPO002_F 39 |
| 22 | JPO002 R 39 |
| 23 | JPO003_F 39 |
| 24 | JPO003_R 39 |

### PCR Reaction Mix:

PrimeSTAR Max DNA polymerase [TaKaRa]
Total 25 μl
1.0 μl Template DNA (1 ng/μl)
9.5 μl H₂O
12.5 μl 2× PrimeSTAR Max pre-mix
1.0 μl Forward primer (5 μM)
1.0 μl Reverse primer (5 μM)

### PCR Program:

98° C./ 2 min
25× (98° C./10 sec, 60° C./15 sec, 72° C./2 min)
10° C./hold

### Example 2: Screening for Higher Productivity by Using 96 MTP Culture

Transformants constructed as in EXAMPLE 1 were fermented in either 96-well MTP (micro titer plate) containing COVE liquid medium (2.0 g/L sucrose, 2.0 g/L iso-maltose, 2.0 g/L maltose, 4.9 mg/L, 0.2 ml/L 5N NaOH, 10 ml/L COVE salt, 10 ml/L 1M acetamide), YPMAc (5 g/L sucrose, 2.5 g/L Yeast extract, 5.0 g/L pepton, 10.0 g/L Soy bean powder, 1.36 g/L CH3COONa 3H2O), at 32° C. for 3 days. Then, glucoamylase activities in culture supernatants were measured at several temperatures by pNPG assay described as follows. The activities are listed in Table 2 and Table 3 as relative activity (yield) to that of AnPav498 which has been used as control.

pNPG Assay

The culture supernatants containing desired enzymes were mixed with same volume of pH 5.0 200 mM NaOAc buffer. Twenty microliter of this mixture was dispensed into either 96-well plate or 8-strip PCR tube. Those samples were mixed with 10 μl of substrate solution containing 0.1% (w/v) pNPG [wako] in pH 5.0 200 mM NaOAc buffer and incubated at 70° C. for 20 min for enzymatic reaction. After the reaction, 60 μl of 0.1M Borax buffer was added to stop the reaction. Eighty microliter of reaction supernatant was taken out and its OD₄₀₅ value was read by photometer to evaluate the enzyme activity.

TABLE 2
List of the relative yield of these variants when
compared with AnPav498 in C2446 which
cultured by Cove-II liquid 1% isomaltose in 96MTP

| host | AnPav498 | JPO001 | JPO003 |
|---|---|---|---|
| C2661 | ND | 136% | 78% |
| C5502 | 185% | 142% | 68% |
| C5503 | 137% | 203% | 107% |

TABLE 3
List of the relative yield of JPO001 in each host compared
with their parents (AnPav498) cultured in Cove-II
liquid medium, and YPMAc medium in 96MTP

| Medium | Host | JPO001 |
|---|---|---|
| COVE-II | C2446 | 123% |
| liquid | C2661 | 185% |
| | C5503 | 141% |
| YPMAc | C2446 | 118% |
| | C2661 | 146% |
| | C5503 | 130% |

### Example 3: Fermentation of the Aspergillus niger in SF

Aspergillus niger strains constructed as in EXAMPLE 1 were fermented on a rotary shaking table in 500 ml baffled flasks containing 100 ml MU1 (260.0 g/L Maltodextrin (MD-11), 3.0 g/L MgSO4 7H2O, 6.0 g/L K2SO4, 5.0 mg/L KH2PO4, 5 ml/L COVE salt) with 4 ml 50% urea at 220 rpm, 30° C. The culture broth was centrifuged (10,000×g, 20 min) and the supernatant was carefully decanted from the precipitates. Then, glucoamylase activities in culture supernatants were measured at several temperatures by pNPG assay described as in EXAMPLE 2. As can be seen in Table 4, the polypeptide yield from JPO001 variant was increased up to 108%, 135% and 151% compared to the polypeptide yield of the AnPav498 control.

TABLE 4
The list of the relative yield of these variants when compared
with their parents (AnPav498 in C2661) which
cultured by MU1 medium in SF with baffle

| | | Relative glucoamylase |
|---|---|---|
| variant | host | activity to control |
| JPO001 | C2661 | 108% |
| JPO001 | C5502 | 135% |
| JPO001 | C5503 | 151% |
| JPO002 | C2661 | 3% |
| JPO002 | C5502 | 75% |
| JPO002 | C5503 | 2% |
| JPO003 | C2661 | 76% |
| JPO003 | C5502 | 92% |
| JPO003 | C5503 | 83% |
| AnPav498 | C2661 | 100% |

### Example 4: Purification of Glucoamylase

Aspergillus niger variant was purified through two steps of ammonium sulfate precipitation and cation exchange chromatography. Finally, the sample was de-salted and buffer exchanged using a centrifugal filter unit (Vivaspin Turbo 15, Sartorius) with 20 mM sodium acetate buffer pH 4.5. Enzyme concentrations were determined by A280 value.

### Example 5: Expression of JPO Variants in A. niger Strains

Expression of JPO variants were tested with A. niger host C5553 harbouring FLP-mediated integration of 3-4 JPO variant copies. FLP-mediated integration has been carried out as described in WO2012/160093. The expression host strain C5553 was isolated by Novozymes and is a derivative of A. niger NN049184 which was isolated from soil described in example 14 in WO2012/160093.

A total of 9 to 10 clones from the same variant were brought to primary evaluation by MTP (Table 5). Compared to the backbone anPAV498, signal modified variant (JPO001) improved the polypeptide activity by 6%. In the secondary evaluation by SF (Table 6) all variants showed significantly increased activity of up to 2414% (construct JPO001, day 6) when compared to the expression with anPAV498.

TABLE 5
The relative glucoamylase activity in MTP fermentation (host: C5553)
relative activity

| | |
|---|---|
| anPAV498 | 100% |
| JPO001 | 106% |
| JPO051 | 89% |
| JPO124 | 84% |

TABLE 6
The relative glucoamylase activity in SF fermentation
Relative activity

| Construct | Day 4 | Day 6 |
|---|---|---|
| AnPAV498 | 100% | 100% |
| JPO001 | 851% | 2414% |
| JPO051-3 | 778% | 2205% |
| JPO051-7 | 770% | 2132% |
| JPO124 | 815% | 2336% |

### Example 6: JPO Variants Test in Lab Tanks

AnPav498 and JPO001 were evaluated in lab-tanks under the current standard conditions in two batches to investigate the effect of signal peptide modification. As results presented in Table 7, compared to AnPav498, JPO001 showed 15% higher titers in C3085 and 77% higher titers in C5553.

TABLE 7
The relative glucoamylase activity in 5 L tank

| | Construct | |
|---|---|---|
| Host | AnPAV498 | JPO001 |
| C3085 | 1 | 1.15 |
| (4copy) | | |
| C5553 | 1 | 1.77 |
| (3copy) | | |

### Example 7: Construction of Plasmid plhar234, pHiTe384 and pHiTe387

The expression plasmids comprising the tandem repeat of nucleotide sequence encoding the R. pusillus alpha-amylase in connection with an Aspergillus promoter, signal sequence JSP001 (plhar234), JSP035 (pHiTe384) and JSP038 (pHiTe387) and terminator, and further comprising an amdS gene for amdS selection in Aspergillus was constructed as follows. The around 1.8 kb region of amylase gene was amplified from a plasmid harboring SEQ ID: NO 84 (described in EP2527448-A1) by PCR with corresponding primer pairs (SEQ ID: 27 and 28 of the present application).

The obtained 1.8 kb DNA fragment was ligated with the BamHI/PmII digest of pHiTe169 (a derivative of pJaL 1470 described in WO2015144936A1) by NEBuilder® HiFi DNA Assembly Master Mix (New England Biolabs) according to the manufacture's protocol, to create single expression plasmid. The resulting plasmids were digested by NheI or NheI/SpeI. Then these fragments derived from the same plasmid were purified by gel extraction kit (QIAGEN) and ligated by the ligation kit (Roche), resulting in the tandem expression plasmids plhar234.

For signal variants JSP035 and JSP038, the overlap extension PCR was used to create the full-length DNA of the alpha-amylase with the signal and leader peptides with corresponding DNA templates and primers.

TABLE 8
PCR amplifications

| Component | Volume | Final Concentration |
|---|---|---|
| 10x Buffer for KOD-Plus- | 5 μl | 1x |
| 2 mM dNTPs | 5 μl | 0.2 mM each |
| 25 mM MgSO₄ | 2 μl | 1.0 mM |
| 10 pmol/μl Primer #1 | 1.5 μl | 0.3 μM |
| 10 pmol/μl Primer #2 | 1.5 μl | 0.3 μM |
| Template DNA | X μl | |
| Genomic DNA Plasmid DNA | 10-200 ng/50 μl | |
| | 1-50 ng/50 μl | |
| PCR grade water | Y μl | |
| KOD-Plus-(1.0 U/μl) | 1 μl | 1.0 U/50 μl |
| (TOYOBO) | | |
| Total reaction volume | 50 μl | |
| 3-step cycle: | | |
| Pre-denaturation: 94° C., 2 min. | | |
| Denaturation: 94 ° C., 15 sec. | | |
| Annealing: Tm-[5-10] °C*, 30 sec. | {close oversize brace} | 35 cycles |
| Extension: 68° C., 1 min./kb | | |

### <1^{st} PCR>

JSP035 template DNA1 (HTJP-1053): SEQ ID NO: 29
JSP035 template DNA2 (HTJP-1149): SEQ ID NO: 30
Forward primer for 1^{st} PCR (HTJP-1183): SEQ ID NO: 31
Reverse primer for 1^{st} PCR (HTJP-1184): SEQ ID NO: 32
JSP038 Template DNA1 (HTJP-1112): SEQ ID NO: 33
JSP038 Template DNA2 (HTJP-1151): SEQ ID NO: 34
Forward primer for 1^{st} PCR (HTJP-1187): SEQ ID NO: 35
Reverse primer for 1^{st} PCR (HTJP-1184): SEQ ID NO: 32

plhar234 was used as DNA template for following PCR:
<JSP035>

Forward primer for 1^{st} PCR (HTJP-1185): SEQ ID NO: 36
Reverse primer for 1^{st} PCR (HTJP-1049): SEQ ID NO: 28
<JSP038>
Forward primer for 1^{st} PCR (HTJP-1186): SEQ ID NO: 37
Reverse primer for 1^{st} PCR (HTJP-1049): SEQ ID NO: 28

The ca. 1.9 kb region of amylase gene with JSP035 was amplified from the 1^{st} PCR fragments by overlap extension PCR with corresponding primer pairs (SEQ ID: 28 and 31).

### <2^{nd} PCR>

Forward primer for 2^{nd} PCR (HTJP-1183): SEQ ID NO: 31
Reverse primer for 2^{nd} PCR (HTJP-1049): SEQ ID NO: 28

The ca. 1.9 kb region of amylase gene with JSP038 was amplified from the 1^{st} PCR fragments by overlap extension PCR with corresponding primer pairs (SEQ ID: 28 and 35).

### <2^{nd} PCR>

Forward primer for 2^{nd} PCR (HTJP-1187): SEQ ID NO: 35
Reverse primer for 2^{nd} PCR (HTJP-1049): SEQ ID NO: 28

The obtained 1.9 kb DNA fragments for both JSP035 and JSP038 was ligated with the BamHI/PmII digest of pHiTe169 by NEBuilder® HiFi DNA Assembly Master Mix (New England Biolabs) according to the manufacture's protocol, to create single expression plasmid. The resulting plasmids were digested by NheI or NheI/SpeI. Then these fragments derived from the same plasmid were purified by gel extraction kit (QIAGEN) and ligated by the ligation kit (Roche), resulting in the tandem expression plasmids pHiTe384 (JSP035) and pHiTe387 (JSP038).

Sequences of signal peptides and Leader peptides (*Bold character is N-terminal end of mature polypeptide of interest) are shown:

according to SEQ ID NO: 41
MRLSTSSLFLSVSLLGKLALG A (JSP001, reference strain)
according to SEQ ID NO: 43
MRLSTSSLFLSVSLLGKLALGFARAPVAAR A (JSP035)
according to SEQ ID NO: 45
MGVSAVLLPLYLLSGVTFGLAFARAPVAAR A (JSP038)

### Example 8: Alpha-Amylase Expression in A. niger Strain

Chromosomal insertion of the R. pusillus alpha-amylase gene with amdS selective marker into A. niger C5554 was performed as described in WO 2012/160093. The R. pusillus alpha-amylase expression plasmids plhar234, pHiTe384 and pHiTe387 should be introduced at four pre-specified loci which are mannosyltransferase (alg2), glucokinase (gukA), acid stable amylase (asaA) and multicopper oxidase (mcoH) by flp recombinase. Strains were purified and subjected to southern blotting analysis to confirm whether the R. pusillus alpha-amylase gene was introduced at mcoH, gukA, asaA and alg2 loci correctly or not. The following set of primers to make non-radioactive probe was used to analyze the selected transformants.

For the promoter region:

| | | |
|---|---|---|
| SEQ ID NO 38: | HTJP-324 | AAGGGATGCAAGACCAAACC |
| SEQ ID NO 39: | HTJP-325 | TGAAGAATTTGTGTTGTCTGAG |

Genomic DNA extracted from the selected transformants was digested by Spel and HindIII, then probed with the promoter region. By the right gene introduction event, hybridized signals at the size of 11.0 kb (alg2), 7.3 kb (mcoH), 11.1 kb (gukA) and 7.8 kb (asaA) by Spel and HindIII digestion was observed probed described above.

### Example 9: Evaluation of the Alpha-Amylase Strains in Lab Tanks

One strain from each signal and leader peptides from C5554 was fermented in lab-scale tanks and their enzyme activities (FAU(F) activities) were measured as described below. The results are shown in the table below (Table 9). The strains with the leader peptide (JSP035 and JSP038) showed around 1.11-1.25 times higher amylase activity than the reference signal without the leader sequence (JSP001) in lab fermenters (Table 9).

TABLE 9
Relative amylase activity in lab tanks

| JSP ID | Strain name | Relative amylase activity |
|---|---|---|
| JSP001 | O73RGP | 1.00 |
| JSP035 | O16MTW | 1.11 |
| JSP038 | O16N4W | 1.17 |
| JSP038 | O16MTS | 1.17 |
| JSP038 | O16MTT | 1.19 |
| JSP038 | O16MTU | 1.25 |

The average FAU(F) activity of the selected six strains from each host strain, wherein the average FAU(F) yields from O73RGP is normalized to 1.00.

Amylase activity was measured as FAU(F) (Fungal a-amylase Units (Fungamyl)), relative to an enzyme standard of a declared strength. Fungamyl is an 1, 4 alpha-D-glucanohydrolyase with the enzyme classification number EC 3.2.1.1. The samples and the alpha-glucosidase in the reagent kit hydrolyze substrate (4,6-ethylidene(G7)-p-nitrophenyl(G1)-alpha, D-maltoheptaoside (ethylidene-G7PNP) to glucose and the yellow-colored p-nitrophenol. The rate of formation of p-nitrophenol can be observed by Konelab (Thermo Fisher Scientific).

TABLE 10
Reaction conditions.

| Parameter | Reaction conditions |
|---|---|
| Temperature [° C.] | 37 |
| pH | 7.00 |
| Substrate conc. | 1.86 mM |
| Enzyme conc. | [0.4576; 2.288] mFAU(F)/mL |
| Reaction time [sec.] | 300 |
| Interval kinetic measuring time | 120 |
| [sec.] | 7 measurements with an interval of 18 sec. |
| Wave length [nm] | 405 |

### Reaction Buffer Composition

87 mM NaCl
52.4 mM HEPES
12.6 mM MgCl2
0.075 mM CaCl2
>4 kU/L alpha-glucosidase

### Substrate Composition

52.4 mM HEPES
22 mM ethylidene-G7PNP

The enzyme activity of the diluted samples is read from the standard curve.

Calculation was conducted as follows:

$$FAU ( F ) / g = \frac{S \times V \times F}{W \times 1000}$$

$$S = Reading from the standard curve in mFAU ( F ) / ml$$

$$V = Volume of the measuring flask used in mL$$

$$F = Dilution factor$$

$$W = Weight of sample in g$$

TABLE 11
Overview of nucleotide and amino acid sequences.

| SEQ ID | Variant, | |
|---|---|---|
| NO: | primer | Description |
| 1 | JPO001 | Nucleotide sequence of leader peptide |
| 2 | JPO001 | Amino acid sequence of leader peptide |
| 3 | JPO001 | Nucleotide sequence of signal peptide |
| 4 | JPO001 | Amino acid sequence of signal peptide |
| 5 | JPO001 | Nucleotide sequence of signal peptide with leader |
| | | peptide |
| 6 | JPO001 | Amino acid sequence of signal peptide with leader |
| | | peptide |
| 7 | AnPav498 | Nucleotide sequence of signal peptide with |
| | | glucoamylase |
| 8 | AnPav498 | Amino acid sequence of signal peptide with |
| | | glucoamylase |
| 9 | JPO001 | Nucleotide sequence of signal peptide with leader |
| | | peptide and glucoamylase |
| 10 | JPO001 | Amino acid sequence of signal peptide with leader |
| | | peptide and glucoamylase |
| 11 | JPO124 | Nucleotide sequence of signal peptide with leader |
| | | peptide and glucoamylase |
| 12 | JPO124 | Amino acid sequence of signal peptide with leader |
| | | peptide and glucoamylase |
| 13 | JPO172 | Nucleotide sequence of signal peptide with leader |
| | | peptide and glucoamylase |
| 14 | JPO172 | Amino acid sequence of signal peptide with leader |
| | | peptide and glucoamylase |
| 15 | AnPav498 | Amino acid sequence of glucoamylase |
| 16 | JPO001 | Amino acid sequence of glucoamylase |
| 17 | JPO124 | Amino acid sequence of glucoamylase |
| 18 | JPO172 | Amino acid sequence of glucoamylase |
| 19 | JPO001 | JPO001_F 42 |
| 20 | JPO001 | JPO001_R 42 |
| 21 | JPO002 | JPO002_F 39 |
| 22 | JPO002 | JPO002_R 39 |
| 23 | JPO003 | JPO003_F 39 |
| 24 | JPO003 | JPO003_R 39 |
| 25 | JPO002 | Amino acid sequence of signal peptide |
| 26 | JPO003 | Amino acid sequence of signal peptide |
| 27 | HTJP-1022 | Forward primer for the JSP001 and amylase |
| 28 | HTJP-1049 | Reverse primer for the JSP001 and amylase |
| 29 | HTJP-1053 | JSP035 template DNA1 |
| 30 | HTJP-1149 | JSP035 template DNA2 |
| 31 | HTJP-1183 | Forward primer for 1^{st} PCR (JSP035) |
| 32 | HTJP-1184 | Reverse primer for 1^{st} PCR (JSP035 and JSP038) |
| 33 | HTJP-1112 | JSP038 Template DNA1 |
| 34 | HTJP-1151 | JSP038 Template DNA2 |
| 35 | HTJP-1187 | Forward primer for 1^{st} PCR (JSP038) |
| 36 | HTJP-1185 | Forward primer for 1^{st} PCR (JSP035) |
| 37 | HTJP-1186 | Forward primer for 1^{st} PCR (JSP038) |
| 38 | HTJP-324 | Primer for the southern probe in the promoter region |
| 39 | HTJP-325 | Primer for the southern probe in the promoter region |
| 40 | JSP001 | Nucleotide sequence of signal peptide |
| | (control) | |
| 41 | JSP001 | Amino acid sequence of signal peptide |
| 42 | JSP035 | Nucleotide sequence of signal peptide with leader |
| | | peptide |
| 43 | JSP035 | Amino acid sequence of signal peptide with leader |
| | | peptide |
| 44 | JSP038 | Nucleotide sequence of signal peptide with leader |
| | | peptide |
| 45 | JSP038 | Amino acid sequence of signal peptide with leader |
| | | peptide |
| 46 | JSP001 | Nucleotide sequence of signal peptide with leader |
| | | peptide and alpha-amylase |
| 47 | JSP001 | Amino acid sequence of signal peptide with leader |
| | | peptide and alpha-amylase |
| 48 | JSP035 | Nucleotide sequence of signal peptide with leader |
| | | peptide and alpha-amylase |
| 49 | JSP035 | Amino acid sequence of signal peptide with leader |
| | | peptide and alpha-amylase |
| 50 | JSP038 | Nucleotide sequence of signal peptide with leader |
| | | peptide and alpha-amylase |
| 51 | JSP038 | Amino acid sequence of signal peptide with leader |
| | | peptide and alpha-amylase |
| 52 | JSP038 | Amino acid sequence of signal peptide |

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

The invention is further defined by the following numbered paragraphs:

1. A fungal host cell comprising in its genome:
a) a first polynucleotide encoding a polypeptide of interest; and
b) a second polynucleotide operably linked in translational fusion to the first polynucleotide upstream of the first polynucleotide, said second polynucleotide encoding a leader peptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO: 2 (FARAPVAAR), preferably the leader peptide is synthetic, or heterologous to the polypeptide of interest.

2. The fungal host cell according to paragraph 1, wherein the leader peptide comprises, consists essentially of, or consists of SEQ ID NO: 2.

3. The fungal host cell according to paragraph 1, wherein the leader peptide is identical to the amino acid sequence of SEQ ID NO: 2.

4. The fungal host cell according to any one of the preceding paragraphs, wherein the host cell comprises in its genome a third polynucleotide encoding a signal peptide, wherein the third polynucleotide is operably linked in translational fusion to the second polynucleotide upstream of the second polynucleotide; and wherein the polypeptide of interest is secreted.

5. The fungal host cell according to paragraph4, wherein the third polynucleotide encodes a signal peptide having a sequence identity of at least 60% e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO: 4, SEQ ID NO: 41 or SEQ ID NO: 52.

6. The fungal host cell according to any one of the preceding paragraphs, wherein the at least one control sequence is operably linked to the signal peptide or the leader peptide, and wherein said control sequence is directing the production of the polypeptide of interest.

7. The fungal host cell according to any one of the preceding paragraphs, wherein the polypeptide of interest is heterologous to the host cell.

8. The fungal host cell according to any one of paragraphs 6 to 7, wherein the at least one control sequence is heterologous to the polynucleotide encoding the polypeptide of interest, the signal peptide, and/or the leader peptide.

9. The fungal host cell according to any one of the preceding paragraphs, wherein the host cell comprises at least two copies of the first and second polynucleotide, such as two, three, four, five or six copies of the first and second polynucleotide.

10. The fungal host cell according to any one of the preceding paragraphs, wherein the second polynucleotide encoding the leader peptide of SEQ ID NO: 2 comprises one or more mutations, preferably nucleotide substitutions, nucleotide deletions or nucleotide insertions. Said mutation(s) leading to a variant of the leader peptide of SEQ ID NO: 2, such as a variant comprising (i) one or more additional amino acids compared to SEQ ID NO:2, (ii) at least one amino acid less compared to SEQ ID NO: 2, e.g. a total of 3 to 8 amino acids, (iii) or an amino acid substitution of at least one amino acid of SEQ ID NO: 2, such as a substitution of the amino acid at a position corresponding to position 1, 2, 3, 4, 5, 6, 7, 8 or 9 of SEQ ID NO: 2.

11. The fungal host cell according to any one of paragraphs 4 to 10, wherein the third polynucleotide encodes a signal peptide having a sequence identity of at least 60% e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO: 4, SEQ ID NO 41 or SEQ ID NO: 52.

12. The fungal host cell according to any one of paragraphs 4 to 11, wherein the third polynucleotide essentially consists of, consists of, or comprises SEQ ID NO: 4, SEQ ID NO: 41 or SEQ ID NO: 52.

13. The fungal host cell according to any one of paragraphs 4 to 11, wherein the third polynucleotide encoding the signal peptide of SEQ ID NO: 4, SEQ ID NO: 41 or SEQ ID NO: 52 comprises one or more mutations, preferably nucleotide substitutions, nucleotide deletions or nucleotide insertions. Said mutation(s) leading to a variant of the signal peptide of SEQ ID NO: 4, SEQ ID NO: 41 or SEQ ID NO: 52, such as a variant comprising (i) one or more additional amino acids compared to SEQ ID NO: 4, SEQ ID NO: 41 or SEQ ID NO: 52, (ii) at least one amino acid less compared to SEQ ID NO: 4, SEQ ID NO: 41 or SEQ ID NO: 52, e.g. a total of 10 to 20 amino acids, (iii) or an amino acid substitution of at least one amino acid of SEQ ID NO: 4, SEQ ID NO: 41 or SEQ ID NO: 52, such as a substitution of the amino acid at a position corresponding to position 1, 2, 3, 4, 5, 6, 7, 8, 9, 10, 11, 12, 13, 14, 15, 16, 17, 18, 19, 20, or 21 of SEQ ID NO: 4, SEQ ID NO: 41 or SEQ ID NO: 52.

14. The fungal host cell according to any one of the preceding paragraphs, wherein the host cell is a yeast host cell; preferably the yeast host cell is selected from the group consisting of Candida, Hansenula, Kluyveromyces, Pichia (Komagataella), Saccharomyces, Schizosaccharomyces, and Yarrowia cell; more preferably the yeast host cell is selected from the group consisting of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica cell, most preferably Pichia pastoris (Komagataella phaffii).

15. The fungal host cell according to any one of paragraphs 1 to 13, wherein the host cell is a filamentous fungal host cell; preferably the filamentous fungal host cell is selected from the group consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma cell; more preferably the filamentous fungal host cell is selected from the group consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride cell; even more preferably the filamentous host cell is selected from the group consisting of Aspergillus oryzae, Fusarium venenatum, and Trichoderma reesei cell; most preferably the filamentous fungal host cell is an Aspergillus niger cell.

16. The fungal host cell according to paragraph 15, wherein the filamentous host cell is an Aspergillus niger cell.

17. The fungal host cell according to paragraph 15, wherein the filamentous host cell is an Aspergillus oryzae cell.

18. The fungal host cell according to paragraph 15, wherein the filamentous host cell is a Trichoderma reesei cell.

19. The fungal host cell according to any one of the preceding paragraphs, wherein the polypeptide of interest comprises an enzyme; preferably the enzyme is selected from the group consisting of hydrolase, isomerase, ligase, lyase, oxidoreductase, or transferase; more preferably an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase.

20. The fungal host cell according to claim 7, wherein the polypeptide of interest is a glycoprotein, preferably an alpha-glucosidase; more preferably an 1,4-alpha-glucosidase; most preferably a glucoamylase, such as a glucoamylase having a sequence identity of at least 60% to SEQ ID NO: 15, SEQ ID NO: 16, SEQ ID NO: 17, SEQ ID NO: 18, SEQ ID NO: 47, SEQ ID NO: 49, or SEQ ID NO: 51.

21. A fungal host cell comprising a polypeptide, said polypeptide comprising a leader peptide operably linked in translational fusion to a polypeptide of interest, wherein the leader peptide has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO: 2 (FARAPVAAR); OR wherein the leader peptide comprises, consists essentially of, or consists of SEQ ID NO: 2.

22. The fungal host cell according to paragraph 21, wherein, the polypeptide further comprises a signal peptide operably linked in translational fusion upstream of the leader peptide having a sequence identity of at least 60% e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO: 4, SEQ ID NO: 41 or SEQ ID NO: 52.

23. The fungal host cell according to any one of paragraphs 21 to 22, wherein the signal peptide upstream of the leader peptide comprises, essentially consists of, or consists of SEQ ID NO: 4, SEQ ID NO: 41 or SEQ ID NO: 52.

24. A method for producing a polypeptide of interest, the method comprising:
i) providing a fungal host cell according to any one of paragraphs 1 to 23,
ii) cultivating said fungal host cell under conditions conducive for expression of the polypeptide of interest; and, optionally
iii) recovering the polypeptide of interest.

25. An isolated or purified polypeptide having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 15, SEQ ID NO: 16, SEQ ID NO: 17, SEQ ID NO: 18, SEQ ID NO: 47, SEQ ID NO: 49, or SEQ ID NO: 51.

26. The isolated or purified polypeptide according to paragraph 25, wherein the polypeptide has glucoamylase activity.

27. The isolated or purified polypeptide according to any one of paragraphs 25 to 26, wherein the polypeptide differs by up to 10 amino acids, e.g., 1, 2, 3, 4, 5, 6, 7, 8, 9, or 10, from the mature polypeptide of SEQ ID NO: 15, SEQ ID NO: 16, SEQ ID NO: 17, SEQ ID NO: 18, SEQ ID NO: 47, SEQ ID NO: 49, or SEQ ID NO: 51.

28. The isolated or purified polypeptide according to any one of paragraphs 25 to 27, wherein the polypeptide comprises, consists essentially of, or consists of the amino acid sequence of SEQ ID NO: 15, SEQ ID NO: 16, SEQ ID NO: 17, SEQ ID NO: 18, SEQ ID NO: 47, SEQ ID NO: 49, or SEQ ID NO: 51.or the mature polypeptide thereof; or is a fragment thereof.

29. The isolated or purified polypeptide according to paragraph 28, wherein the mature polypeptide is identical to SEQ ID NO: 15.

30. The isolated or purified polypeptide according to paragraph 28, wherein the mature polypeptide is identical to SEQ ID NO: 16.

31. The isolated or purified polypeptide according to paragraph 28, wherein the mature polypeptide is identical to SEQ ID NO: 17.

32. The isolated or purified polypeptide according to paragraph 28, wherein the mature polypeptide is identical to SEQ ID NO: 18.

33. An isolated polynucleotide encoding a signal peptide comprising, essentially consisting of, or consisting of amino acids 1 to 21 of SEQ ID NO: 4, SEQ ID NO: 41 or SEQ ID NO: 52, amino acids 1 to 21 of SEQ ID NO: 6 or amino acids 1 to 21 of SEQ ID NO: 10.

34. An isolated polynucleotide encoding a synthetic leader peptide comprising, essentially consisting of, or consisting of amino acids 1 to 9 of SEQ ID NO: 2, amino acids 22 to 30 of SEQ ID NO: 6 or amino acids 22 to 30 of SEQ ID NO: 10.

35. The isolated polynucleotide of paragraph 34, wherein the polynucleotide encoding the leader peptide of SEQ ID NO: 2 comprises one or more mutations, preferably nucleotide substitutions, nucleotide deletions or nucleotide insertions.

36. The isolated polynucleotide of paragraph 35, wherein said mutation(s) are resulting in a variant of the signal peptide of SEQ ID NO: 2, such as a variant comprising (i) one or more additional amino acids compared to SEQ ID NO: 2, (ii) at least one amino acid less compared to SEQ ID NO: 2, e.g. a total of 4 to 8 amino acids, (iii) or an amino acid substitution of at least one amino acid of SEQ ID NO: 2, such as a substitution of the amino acid at a position corresponding to position 1, 2, 3, 4, 5, 6, 7, 8, or 9 of SEQ ID NO: 2.

37. The isolated polynucleotide according to any one of paragraphs 34 to 36, wherein the polynucleotide is encoding a leader peptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO: 2 (FARAPVAAR).

38. The isolated polynucleotide according to paragraph 37, wherein the leader peptide is identical to SEQ ID NO: 2.

39. An isolated polynucleotide encoding a signal peptide and a leader peptide comprising, essentially consisting of, or consisting of amino acids 1 to 30 of SEQ ID NO: 6 or amino acids 1 to 30 of SEQ ID NO: 10, SEQ ID NO: 43 or SEQ ID NO: 45.

40. The isolated polynucleotide according to paragraph 39, wherein the polynucleotide is encoding a signal peptide and a leader peptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to

SEQ ID NO: 6
(MRLTLLSGVAGVLCAGQLTAAFARAPVAAR),
SEQ ID NO: 43
(MRLSTSSLFLSVSLLGKLALGFARAPVAAR);
or
SEQ ID NO: 45
(MGVSAVLLPLYLLSGVTFGLAFARAPVAAR).

41. The isolated polynucleotide according to any one of paragraphs 33 to 40, wherein the polynucleotide encoding the signal peptide or leader peptide is operably linked in translational fusion to a gene encoding a protein, such as a glucoamylase.

42. A nucleic acid construct comprising a first polynucleotide encoding a polypeptide of interest, and a second polynucleotide operably linked to the first polynucleotide encoding a leader peptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO: 2 (FARAPVAAR), preferably the leader peptide is synthetic, or heterologous to the polypeptide of interest.

43. The nucleic acid construct according to paragraph 42, wherein the leader peptide comprises, consists essentially of, or consists of SEQ ID NO: 2.

44. The nucleic acid construct according to paragraph 43, wherein the second polynucleotide encoding the leader peptide of SEQ ID NO: 2 comprises one or more mutations, preferably nucleotide substitutions, nucleotide deletions or nucleotide insertions.

45. The nucleic acid construct according to paragraph 44, wherein said mutation(s) is/are resulting in a variant of the leader peptide of SEQ ID NO: 2, such as a variant comprising (i) one or more additional amino acids compared to SEQ ID NO: 2, (ii) at least one amino acid less compared to SEQ ID NO: 2, e.g. a total of 4 to 8 amino acids, (iii) or an amino acid substitution of at least one amino acid of SEQ ID NO: 2, such as a substitution of the amino acid at a position corresponding to position 1, 2, 3, 4, 5, 6, 7, 8, or 9 of SEQ ID NO: 2.

46. The nucleic acid construct according to any one of paragraphs 42 to 45, wherein the second polynucleotide is operably linked to one or more control sequences that direct the production of the polypeptide in an expression host.

47. The nucleic acid construct according to any one of paragraphs 42 to 46, wherein the nucleic acid construct comprises a third polynucleotide encoding a signal peptide, wherein the third polynucleotide is operably linked in translational fusion to the second polynucleotide upstream of the second polynucleotide; and the signal peptide having a sequence identity of at least 60% e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, to SEQ ID NO: 4, SEQ ID NO: 41 or SEQ ID NO: 52.

48. The nucleic acid construct according to paragraph 47, wherein the signal peptide consists of, essentially consists of, or comprises SEQ ID NO: 4, SEQ ID NO: 41 or SEQ ID NO: 52.

49. The nucleic acid construct according to any one of paragraphs 46 to 47, wherein the third polynucleotide is operably linked to one or more control sequences that direct the production of the polypeptide in an expression host

50. The nucleic acid construct according to any one of paragraphs 47 to 49, wherein the third polynucleotide encoding the signal peptide comprises one or more mutations, preferably nucleotide substitutions, nucleotide deletions or nucleotide insertions.

51. The nucleic acid construct according to paragraph 50, wherein said mutation(s) are resulting in a variant of the signal peptide of SEQ ID NO: 4, SEQ ID NO: 41 or SEQ ID NO: 52, such as a variant comprising (i) one or more additional amino acids compared to SEQ ID NO: 4, SEQ ID NO: 41 or SEQ ID NO: 52 (ii) at least one amino acid less compared to SEQ ID NO: 4, SEQ ID NO: 41 or SEQ ID NO: 52, e.g. a total of 10 to 20 amino acids, (iii) or an amino acid substitution of at least one amino acid of SEQ ID NO: 4, SEQ ID NO: 41 or SEQ ID NO: 52, such as a substitution of the amino acid at a position corresponding to position 1, 2, 3, 4, 5, 6, 7, 8, 9, 10, 11, 12, 13, 14, 15, 16, 17, 18, 19, 20, or 21 of SEQ ID NO: 4, SEQ ID NO: 41 or SEQ ID NO: 52.

52. An expression vector comprising a polynucleotide or nucleic acid construct according to any one of paragraphs 33 to 51.

53. A fungal host cell comprising a polynucleotide, a nucleic acid construct or an expression vector according to any one of paragraphs 33 to 52.

54. An isolated or purified polypeptide having glucoamylase activity, selected from the group consisting of:
(a) a polypeptide having at least 60% sequence identity to SEQ ID NO: 15, SEQ ID NO: 16, SEQ ID NO: 17, SEQ ID NO: 18, SEQ ID NO: 47, SEQ ID NO: 49, or SEQ ID NO: 51;
(b) a polypeptide encoded by a polynucleotide that hybridizes under medium stringency conditions with the full-length complement of the mature polypeptide coding sequence of SEQ ID NO: 7, SEQ ID NO: 9, SEQ ID NO: 11, SEQ ID NO: 13, SEQ ID NO: 46, SEQ ID NO: 48, or SEQ ID NO: 50;
(c) a polypeptide encoded by a polynucleotide having at least 60% sequence identity to the mature polypeptide coding sequence of SEQ ID NO: 7, SEQ ID NO: 9, SEQ ID NO: 11, SEQ ID NO: 13, SEQ ID NO: 46, SEQ ID NO: 48, or SEQ ID NO: 50;
(d) a polypeptide derived from a mature polypeptide of SEQ ID NO: 15, SEQ ID NO: 16, SEQ ID NO: 17, SEQ ID NO: 18, SEQ ID NO: 47, SEQ ID NO: 49, or SEQ ID NO: 51, by substitution, deletion or addition of one or several amino acids in the mature polypeptide of SEQ ID NO: 15, SEQ ID NO: 16, SEQ ID NO: 17, SEQ ID NO: 18, SEQ ID NO: 47, SEQ ID NO: 49, or SEQ ID NO: 51; and
(e) a fragment of the polypeptide of (a), (b), (c), or (d) that has glucoamylase activity.

55. An isolated or purified polypeptide having glucoamylase activity, which is:
(a) a polypeptide having at least 60% sequence identity to SEQ ID NO: 15, SEQ ID NO: 16, SEQ ID NO: 17, SEQ ID NO: 18, SEQ ID NO: 47, SEQ ID NO: 49, or SEQ ID NO: 51; or
(b) a fragment of the polypeptide of (a), that has glucoamylase activity.

56. The polypeptide of any one of paragraphs 54 to 55, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 15, SEQ ID NO: 16, SEQ ID NO: 17, SEQ ID NO: 18, SEQ ID NO: 47, SEQ ID NO: 49, or SEQ ID NO: 51.

57. The polypeptide of any one of paragraphs 54 -56, which is encoded by a polynucleotide that hybridizes under medium stringency conditions, medium-high stringency conditions, high stringency conditions, or very high stringency conditions with the full-length complement of the mature polypeptide coding sequence of SEQ ID NO: 7, SEQ ID NO: 9, SEQ ID NO: 11, SEQ ID NO: 13, SEQ ID NO: 46, SEQ ID NO: 48, or SEQ ID NO: 50.

58. The polypeptide of any one of paragraphs 54-57, which is encoded by a polynucleotide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 7, SEQ ID NO: 9, SEQ ID NO: 11, SEQ ID NO: 13, SEQ ID NO: 46, SEQ ID NO: 48, or SEQ ID NO: 50.

59. The polypeptide of any one of paragraphs 54-58, which is a variant of SEQ ID NO: 15, SEQ ID NO: 16, SEQ ID NO: 17, SEQ ID NO: 18, SEQ ID NO: 47, SEQ ID NO: 49, or SEQ ID NO: 51, comprising a substitution, deletion, and/or insertion at one or more positions.

60. The polypeptide of any one of paragraphs 54-59, comprising, consisting essentially of, or consisting of SEQ ID NO: 15, SEQ ID NO: 16, SEQ ID NO: 17, SEQ ID NO: 18, SEQ ID NO: 47, SEQ ID NO: 49, or SEQ ID NO: 51.

61. The polypeptide of any one of paragraphs 54-60, comprising SEQ ID NO: 15, SEQ ID NO: 16, SEQ ID NO: 17, SEQ ID NO: 18, SEQ ID NO: 47, SEQ ID NO: 49, or SEQ ID NO: 51 and an N-terminal extension and/or C-terminal extension of 1-10 amino acids, e.g., 1, 2, 3, 4, 5, 6, 7, 8, 9, or 10 amino acids.

62. The polypeptide according to paragraph 61, comprising a leader peptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 2.

63. A fusion polypeptide comprising the polypeptide of any one of paragraphs 54 to 62 and a second polypeptide.

64. A granule, which comprises:
(a) a core comprising the polypeptide of any one of paragraphs 54 to 63, and optionally,
(b) a coating consisting of one or more layer(s) surrounding the core.

65. A granule, which comprises:
(a) a core, and
(b) a coating consisting of one or more layer(s) surrounding the core, wherein the coating comprises the polypeptide of any one of paragraphs 54 to 63.

66. A composition comprising the polypeptide of any one of paragraphs 54 to 63 or the granule of paragraph 64 or 65.

67. A whole broth formulation or cell culture composition comprising the polypeptide of any one of paragraphs 54 to 63.

68. An isolated or purified polynucleotide encoding the polypeptide of any one of paragraphs 54 to 63.

1-17. (canceled)

18. An Aspergillus host cell comprising in its genome:
a) a first polynucleotide encoding a polypeptide of interest; and
b) a second polynucleotide operably linked in translational fusion to the first polynucleotide upstream of the first polynucleotide, said second polynucleotide encoding a leader peptide having a sequence identity of at least 80% to SEQ ID NO: 2 (FARAPVAAR),
wherein the host cell comprises in its genome a third polynucleotide encoding a signal peptide,
wherein the third polynucleotide is operably linked in translational fusion to the second polynucleotide upstream of the second polynucleotide, and
wherein the leader peptide is heterologous to the signal peptide.

19. The Aspergillus host cell according to claim 18, wherein the leader peptide has a sequence identity of at least 90% to SEQ ID NO: 2 (FARAPVAAR).

20. The Aspergillus host cell according to claim 18, wherein the leader peptide has a sequence identity of at least 95% to SEQ ID NO: 2 (FARAPVAAR).

21. The Aspergillus host cell according to claim 18, wherein the leader peptide has a sequence identity of at least 98% to SEQ ID NO: 2 (FARAPVAAR).

22. The Aspergillus host cell according to claim 18, wherein the leader peptide is synthetic, or heterologous to the polypeptide of interest.

23. The Aspergillus host cell according to claim 18, wherein the leader peptide comprises, consists essentially of, or consists of SEQ ID NO: 2.

24. The Aspergillus host cell according to claim 18, wherein the polypeptide of interest is secreted.

25. The Aspergillus host cell according to claim 18, wherein the third polynucleotide encodes a signal peptide having a sequence identity of at least 80% to SEQ ID NO: 4, SEQ ID NO: 41 or SEQ ID NO: 52.

26. The Aspergillus host cell according to claim 18, wherein the third polynucleotide encodes a signal peptide having a sequence identity of at least 90% to SEQ ID NO: 4, SEQ ID NO: 41 or SEQ ID NO: 52.

27. The Aspergillus host cell according to claim 18, wherein the third polynucleotide encodes a signal peptide having a sequence identity of at least 95% to SEQ ID NO: 4, SEQ ID NO: 41 or SEQ ID NO: 52.

28. The Aspergillus host cell according to claim 18, wherein the third polynucleotide encodes a signal peptide having a sequence identity of at least 98% to SEQ ID NO: 4, SEQ ID NO: 41 or SEQ ID NO: 52.

29. The Aspergillus host cell according to claim 18, wherein the third polynucleotide encodes a signal peptide comprises, consists essentially of, or consists of SEQ ID NO: 4, SEQ ID NO: 41 or SEQ ID NO: 52.

30. The Aspergillus host cell according to claim 18, wherein the Aspergillus host cell is selected from the group consisting of Aspergillus awamori, Aspergillus foetidus, Aspergillus fumigatus, Aspergillus japonicus, Aspergillus nidulans , Aspergillus niger, and Aspergillus oryzae.

31. The Aspergillus host cell according to claim 18, wherein the Aspergillus host cell is an Aspergillus oryzae cell.

32. The Aspergillus host cell according to claim 18, wherein the Aspergillus host cell is an Aspergillus niger cell.

33. The Aspergillus host cell according to claim 18, wherein the polypeptide of interest is an enzyme.

34. The Aspergillus host cell according to claim 33, wherein the enzyme is selected from the group consisting of an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase.

35. The Aspergillus host cell according to claim 18, wherein the polypeptide of interest is a glucoamylase.

36. A method for producing a polypeptide of interest, the method comprising:
i) providing an Aspergillus host cell according to claim 18, and
ii) cultivating said Aspergillus host cell under conditions conducive for expression of the polypeptide of interest.

37. The method of claim 36 further comprising:
iii) recovering the polypeptide of interest.


SEQUENCE LISTING (52 sequences)

SEQ ID NO: 1 - DNA, 27 bp, Artificial Sequence
  JPO001 leader peptide
ttcgcacgtgcacctgttgctgctaga

SEQ ID NO: 2 - protein, 9 aa, Artificial Sequence
  JPO001 leader peptide
FARAPVAAR

SEQ ID NO: 3 - DNA, 63 bp, Artificial Sequence
  JPO001 signal peptide cDNA
atgcgtctcactctattatcaggtgtagccggcgttctctgcgcaggacagctgacggcggcg

SEQ ID NO: 4 - protein, 21 aa, Penicillium oxalicum
MRLTLLSGVAGVLCAGQLTAA

SEQ ID NO: 5 - DNA, 90 bp, Artificial Sequence
  JPO001 signal peptide with leader peptide
atgcgtctcactctattatcaggtgtagccggcgttctctgcgcaggacagctgacggcggcgttcgcacgtgcacctgttgctgctaga

SEQ ID NO: 6 - protein, 30 aa, Artificial Sequence
  JPO001 signal peptide with leader peptide
MRLTLLSGVAGVLCAGQLTAAFARAPVAAR

SEQ ID NO: 7 - DNA, 1851 bp, Artificial Sequence
  AnPav498 signal peptide with glucoamylase cDNA
atgcgtctcactctattatcaggtgtagccggcgttctctgcgcaggacagctgacggcggcgcgtaacgattcgaagggtgggaatctgacgttcttcatccacaaagagggcgagcggtcgctccaaggcatcttggacaatctcggtgggcgaggtaagaaaacacccggcactgccgcagggttgtttattgccagtccaaacacagagaatccaaactattattatacatggactcgtgactcagctttggccgccaagtgcttgatcgacctgttcgaagactctcgggcagtctttccaattgaccgcaaatacttggaaacaggaattcgggactacgtgtcgtcccaagcaatcctccagagtgtgtctaatccttctggaaccctgaaggatggctctggtctgggtgaacccaagtttgagattgacctgaatcccttttcgggtgcctggggtcggcctcagcgggatggcccagcgctgcgagcgaccgctatgatcacctacgccaactacctgatatcccatggtcagaaatcggatgtgtcacaggtcatgtggccgattattgccaatgatctagcatatgttggtcaatactggaataataccggatttgacctgtgggaagaggtggatgggtcaagctttttcacgattgcggtccagcaccgagcccttgttgaaggctcgcaactggcgaaaaagctcggcaagtcctgcgatgcctgtgattctcagcctccccagatattgtgtttcctgcagagtttctggaacggaaagtacatcacctccaacatcaacacgcaagcaagccgctctggtatcgacctggactctgtcctgggaagcattcatacctttgatcccgaagcagcctgtgacgatgcaactttccagccttgttctgcccgcgctctggcgaaccacaaggtctatgtggattccttccgctctatctacaagattaatgcgggtcttgcagagggatcggctgccaacgttggccgctaccccgaggatgtttacttcggaggcaatccatggtatctcgccaccctaggcgcatctgaattgctttacgacgccttgtaccagtgggacagacttggcaaacttgaagtctcggagacctcgttgtcattcttcaaagactttgacgcgaccgtgaaaattggctcgtactcgaggaacagcaagacctacaagaaattgacccagtccatcaagtcgtacgcggacgggttcatccagttagtgcagcagtacactccttctaatggatctctggccgagcaatacgatcgcaatacggctgctcctctctctgcaaacgatctgacttggtcatttgcctctttcttgacggctacgcaacgccgcgatgccgtggttcctccctcctggggcgcaaagtcggcaaacaaagtcccaaccacttgttcagcctcccctgttgtgggtacttataaggcgcccacggcaactttctcatccaagactaagtgcgtccccgctaaagatattgtgcctatcacgttctacctgattgagaacacttactatggagagaacgtcttcatgagtggcaacattactgcgctgggtaactgggacgccaagaaaggcttcccactcaccgcaaacctctacacgcaagatcaaaacttgtggttcgccagtgtcgagttcatcccagcaggcacaccctttgagtacaagtactacaaggtcgagcccaatggcgatattacttgggagaagggtcccaaccgggtgttcgtcgctcccacgggatgcccagttcagcctcactccaacgacgtgtggcagttttga

SEQ ID NO: 8 - protein, 616 aa, Penicillium oxalicum
MRLTLLSGVAGVLCAGQLTAARNDSKGGNLTFFIHKEGERSLQGILDNLGGRGKKTPGTAAGLFIASPNTENPNYYYTWTRDSALAAKCLIDLFEDSRAVFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 9 - DNA, 1878 bp, Artificial Sequence
  JPO001 signal peptide with leader peptide and glucoamylase
atgcgtctcactctattatcaggtgtagccggcgttctctgcgcaggacagctgacggcggcgttcgcacgtgcacctgttgctgctagagccaacgattcgaagggtgggaatctgacgttcttcatccacaaagagggcgagcggtcgctccaaggcatcttggacaatctcggtgggcgaggtaagaaaacacccggcactgccgcagggttgtttattgccagtccaaacacagagaatccaaactattattatacatggactcgtgactcagctttggccgccaagtgcttgatcgacctgttcgaagactctcgggcagtctttccaattgaccgcaaatacttggaaacaggaattcgggactacgtgtcgtcccaagcaatcctccagagtgtgtctaatccttctggaaccctgaaggatggctctggtctgggtgaacccaagtttgagattgacctgaatcccttttcgggtgcctggggtcggcctcagcgggatggcccagcgctgcgagcgaccgctatgatcacctacgccaactacctgatatcccatggtcagaaatcggatgtgtcacaggtcatgtggccgattattgccaatgatctagcatatgttggtcaatactggaataataccggatttgacctgtgggaagaggtggatgggtcaagctttttcacgattgcggtccagcaccgagcccttgttgaaggctcgcaactggcgaaaaagctcggcaagtcctgcgatgcctgtgattctcagcctccccagatattgtgtttcctgcagagtttctggaacggaaagtacatcacctccaacatcaacacgcaagcaagccgctctggtatcgacctggactctgtcctgggaagcattcatacctttgatcccgaagcagcctgtgacgatgcaactttccagccttgttctgcccgcgctctggcgaaccacaaggtctatgtggattccttccgctctatctacaagattaatgcgggtcttgcagagggatcggctgccaacgttggccgctaccccgaggatgtttacttcggaggcaatccatggtatctcgccaccctaggcgcatctgaattgctttacgacgccttgtaccagtgggacagacttggcaaacttgaagtctcggagacctcgttgtcattcttcaaagactttgacgcgaccgtgaaaattggctcgtactcgaggaacagcaagacctacaagaaattgacccagtccatcaagtcgtacgcggacgggttcatccagttagtgcagcagtacactccttctaatggatctctggccgagcaatacgatcgcaatacggctgctcctctctctgcaaacgatctgacttggtcatttgcctctttcttgacggctacgcaacgccgcgatgccgtggttcctccctcctggggcgcaaagtcggcaaacaaagtcccaaccacttgttcagcctcccctgttgtgggtacttataaggcgcccacggcaactttctcatccaagactaagtgcgtccccgctaaagatattgtgcctatcacgttctacctgattgagaacacttactatggagagaacgtcttcatgagtggcaacattactgcgctgggtaactgggacgccaagaaaggcttcccactcaccgcaaacctctacacgcaagatcaaaacttgtggttcgccagtgtcgagttcatcccagcaggcacaccctttgagtacaagtactacaaggtcgagcccaatggcgatattacttgggagaagggtcccaaccgggtgttcgtcgctcccacgggatgcccagttcagcctcactccaacgacgtgtggcagttttga

SEQ ID NO: 10 - protein, 625 aa, Artificial Sequence
  JPO001 signal peptide with leader peptide and glucoamylase
MRLTLLSGVAGVLCAGQLTAAFARAPVAARANDSKGGNLTFFIHKEGERSLQGILDNLGGRGKKTPGTAAGLFIASPNTENPNYYYTWTRDSALAAKCLIDLFEDSRAVFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 11 - DNA, 1878 bp, Artificial Sequence
  JPO124 signal peptide with leader peptide and glucoamylase
atgcgtctcactctattatcaggtgtagccggcgttctctgcgcaggacagctgacggcggcgttcgcacgtgcacctgttgctgctagagccaacgattcgaagagtacgaatctgacgttcttcatccacaaagagggcgagcggtcgctccaaggcatcttggacaatctcggtgggttcggtaagtacacacccggcactgccgcagggttgtttattgccagtccaaacacagagaatccaaactattattatacatggactcgtgactcagctttggccgccaagtgcttgatcgacctgttcgaagactctcgggcagtctttccaattgaccgcaaatacttggaaacaggaattcgggactacgtgtcgtcccaagcaatcctccagaacgtgtctaatccctctggaaccctgaaggatggctctggtctgggtgaacccaagtttgagattgacctgaatcccttttcgggtccctggggtcggcctcagcgggatggcccagcgctgcgagcgaccgctatgatcacctacgccaactacctgatatcccatggtcagaaatcggatgtgtcacaggtcatgtggccgattattgccaatgatctagcatatgttggtcaatactggaataataccggatttgacctgtgggaagaggtggatgggtcaagctttttcacgattgcggtccagcaccgagcccttgttgaaggctcgcaactggcgaaaaagctcggcaagtcctgcgatgcctgtgattctcagcctccccagatattgtgtttcctgcagagtttctggaacggaaagtacatcacctccaacatcaacacgcaagcaagccgctctggtatcgacctggactctgtcctgggaagcattcatacctttgatcccgaagcagcctgtgacgatgcaactttccagccttgttctgcccgcgctctggcgaaccacaaggtctatgtggattccttccgctctatctacaagattaatgcgggtcttgcagagggatcggctgccaacgttggccgctaccccgaggatgtttacttcggaggcaatccatggtatctcgccaccctaggcgcatctgaattgctttacgacgccttgtaccagtgggacagacttggcaaacttgaagtctcggagacctcgttgtcattcttcaaagactttgacgcgaccgtgaaaattggctcgtactcgaggaacagcaagacctacaagaaattgacccagtccatcaagtcgtacgcggacgggttcatccagttagtgcagcagtacactccttctaatggatctctggccgagcaatacgatcgcaatacggctgctcctctctctgcaaacgatctgacttggtcatttgcctctttcttgacggctacgcaacgccgcaatgcctcggttcctccctcctggggcgcaaagtcggcaaacaaagtcccaaccacttgttcagcctcccctgttgtgggtacttataaggcgcccacggcaactttcccatccaagactaagtgcgtccccgctaaagatattgtgcctatcacgttctacctgattgagaacacttactatggagagaacgtcttcatgagtggcaacattactgcgctgggtaactgggacgccaagaaaggcttcccactcaccgcaaacctctacacgcaagatcaaaacttgtggttcgccagtgtcgagttcatcccagcaggcacaccctttgagtacaagtactacaaggtcgagcccaatggcactattgtttgggagaagggtcccaaccgggtgttcgtcgctcccacgggatgcccagttcagcctcactccaacgacgtgtggcgctcctga

SEQ ID NO: 12 - protein, 625 aa, Artificial Sequence
  JPO124 signal peptide with leader peptide and glucoamylase
MRLTLLSGVAGVLCAGQLTAAFARAPVAARANDSKSTNLTFFIHKEGERSLQGILDNLGGFGKYTPGTAAGLFIASPNTENPNYYYTWTRDSALAAKCLIDLFEDSRAVFPIDRKYLETGIRDYVSSQAILQNVSNPSGTLKDGSGLGEPKFEIDLNPFSGPWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRNASVPPSWGAKSANKVPTTCSASPVVGTYKAPTATFPSKTKCVPAKDIVPITFYLIENTYYGENVFMSGNITALGNWDAKKGFPLTANLYTQDQNLWFASVEFIPAGTPFEYKYYKVEPNGTIVWEKGPNRVFVAPTGCPVQPHSNDVWRS

SEQ ID NO: 13 - DNA, 1878 bp, Artificial Sequence
  JPO172 signal peptide with leader peptide and glucoamylase
atgcgtctcactctattatcaggtgtagccggcgttctctgcgcaggacagctgacggcggcgttcgcacgtgcacctgttgctgctagagccaacgattcgaagagtacgaatctgacgttcttcatccacaaagagggcgagcggtcgctccaaggcatcttggacaatctcggtgggttcggtaagtacacacccggcactgccgcagggttgtttattgccagtccaaacacacggaatccaaactattattatacatggactcgtgactcagctttggccgccaagtgcttgatcgacctgttcgaagactctcgggcagtctttccaattgaccgcaaatacttggaaacaggaattcgggactacgtgtcgtcccaagcaatcctccagaacgtgtctaatccctctggaaccctgaaggatggctctggtctgggtgaacccaagtttgagattgacctgaatcccttttcgggtccctggggtcggcctcagcgggatggcccagcgctgcgagcgaccgctatgatcacctacgccaactacctgatatcccatggtcagaaatcggatgtgtcacaggtcatgtggccgattattgccaatgatctagcatatgttggtcaatactggaataataccggatttgacctgtgggaagaggtggatgggtcaagctttttcacgattgcggtccagcaccgagcccttgttgaaggctcgcaactggcgaaaaagctcggcaagtcctgcgatgcctgtgattctcagcctccccagatattgtgtttcctgcagagtttctggaacggaaagtacatcacctccaacatcaacacgcaagcaagccgctctggtatcgacctggactctgtcctgggaagcattcatacctttgatcccgaagcagcctgtgacgatgcaactttccagccttgttctgcccgcgctctggcgaaccacaaggtctatgtggattccttccgctctatctacaagattaatgcgggtcttgcagagggatcggctgccaacgttggccgctaccccgaggatgtttacttcggaggcaatccatggtatctcgccaccctaggcgcatctgaattgctttacgacgccttgtaccagtgggacagacttggcaaacttgaagtctcggagacctcgttgtcattcttcaaagactttgacgcgaccgtgaaaattggctcgtactcgaggaacagcaagacctacaagaaattgacccagtccatcaagtcgtacgcggacgggttcatccagttagtgcagcagtacactccttctaatggatctctggccgagcaatacgatcgcaatacggctgctcctctctctgcaaacgatctgacttggtcatttgcctctttcttgacggctacgcaacgccgcaatgcctcggttcctccctcctggggcgcaaagtcggcaaacaaagtcccaaccacttgttcagcctcccctgttgtgggtacttataaggcgcccacggcaactttcccatccaagcctaagtgcgtccccgctaaagatattgtgcctatcacgttctacctgattgccaacacttactatggagagaacgtcttcatgagtggcaacattactgcgctgggtaactgggacgccaagaaaggcttcccactcaccgcaaacctctacacgcaagatcaacccttgtggttcgccagtgtcgagttcatcccagcaggcacaccctttgagtacaagtactacaaggtcgagcccaatggcactattgtttgggagaagggtcccaaccgggtgttcgtcgctcccacgggatgcccagttcagcctcactccaacgacgtgtggcgctcctga

SEQ ID NO: 14 - protein, 625 aa, Artificial Sequence
  JPO172 signal peptide with leader peptide and glucoamylase
MRLTLLSGVAGVLCAGQLTAAFARAPVAARANDSKSTNLTFFIHKEGERSLQGILDNLGGFGKYTPGTAAGLFIASPNTRNPNYYYTWTRDSALAAKCLIDLFEDSRAVFPIDRKYLETGIRDYVSSQAILQNVSNPSGTLKDGSGLGEPKFEIDLNPFSGPWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRNASVPPSWGAKSANKVPTTCSASPVVGTYKAPTATFPSKPKCVPAKDIVPITFYLIANTYYGENVFMSGNITALGNWDAKKGFPLTANLYTQDQPLWFASVEFIPAGTPFEYKYYKVEPNGTIVWEKGPNRVFVAPTGCPVQPHSNDVWRS

SEQ ID NO: 15 - protein, 595 aa, Penicillium oxalicum
RNDSKGGNLTFFIHKEGERSLQGILDNLGGRGKKTPGTAAGLFIASPNTENPNYYYTWTRDSALAAKCLIDLFEDSRAVFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 16 - protein, 595 aa, Artificial Sequence
  JPO001 glucoamylase variant
ANDSKGGNLTFFIHKEGERSLQGILDNLGGRGKKTPGTAAGLFIASPNTENPNYYYTWTRDSALAAKCLIDLFEDSRAVFPIDRKYLETGIRDYVSSQAILQSVSNPSGTLKDGSGLGEPKFEIDLNPFSGAWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRDAVVPPSWGAKSANKVPTTCSASPVVGTYKAPTATFSSKTKCVPAKDIVPITFYLIENTYYGENVFMSGNITALGNWDAKKGFPLTANLYTQDQNLWFASVEFIPAGTPFEYKYYKVEPNGDITWEKGPNRVFVAPTGCPVQPHSNDVWQF

SEQ ID NO: 17 - protein, 595 aa, Artificial Sequence
  JPO124 glucoamylase variant
ANDSKSTNLTFFIHKEGERSLQGILDNLGGFGKYTPGTAAGLFIASPNTENPNYYYTWTRDSALAAKCLIDLFEDSRAVFPIDRKYLETGIRDYVSSQAILQNVSNPSGTLKDGSGLGEPKFEIDLNPFSGPWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRNASVPPSWGAKSANKVPTTCSASPVVGTYKAPTATFPSKTKCVPAKDIVPITFYLIENTYYGENVFMSGNITALGNWDAKKGFPLTANLYTQDQNLWFASVEFIPAGTPFEYKYYKVEPNGTIVWEKGPNRVFVAPTGCPVQPHSNDVWRS

SEQ ID NO: 18 - protein, 595 aa, Artificial Sequence
  JPO172 glucoamylase variant
ANDSKSTNLTFFIHKEGERSLQGILDNLGGFGKYTPGTAAGLFIASPNTRNPNYYYTWTRDSALAAKCLIDLFEDSRAVFPIDRKYLETGIRDYVSSQAILQNVSNPSGTLKDGSGLGEPKFEIDLNPFSGPWGRPQRDGPALRATAMITYANYLISHGQKSDVSQVMWPIIANDLAYVGQYWNNTGFDLWEEVDGSSFFTIAVQHRALVEGSQLAKKLGKSCDACDSQPPQILCFLQSFWNGKYITSNINTQASRSGIDLDSVLGSIHTFDPEAACDDATFQPCSARALANHKVYVDSFRSIYKINAGLAEGSAANVGRYPEDVYFGGNPWYLATLGASELLYDALYQWDRLGKLEVSETSLSFFKDFDATVKIGSYSRNSKTYKKLTQSIKSYADGFIQLVQQYTPSNGSLAEQYDRNTAAPLSANDLTWSFASFLTATQRRNASVPPSWGAKSANKVPTTCSASPVVGTYKAPTATFPSKPKCVPAKDIVPITFYLIANTYYGENVFMSGNITALGNWDAKKGFPLTANLYTQDQPLWFASVEFIPAGTPFEYKYYKVEPNGTIVWEKGPNRVFVAPTGCPVQPHSNDVWRS

SEQ ID NO: 19 - DNA, 42 bp, Artificial Sequence
  Primer JPO001_F 42
cgtgcacctgttgctgctagagccaacgattcgaagggtggg

SEQ ID NO: 20 - DNA, 42 bp, Artificial Sequence
  Primer JPO001 R 42
agcagcaacaggtgcacgtgcgaacgccgccgtcagctgtcc

SEQ ID NO: 21 - DNA, 39 bp, Artificial Sequence
  Primer JPO002 F 39
aagagaaccggattggcttatgcgcgtaacgattcgaag

SEQ ID NO: 22 - DNA, 39 bp, Artificial Sequence
  Primer JPO002 R 39
ataagccaatccggttctctttcctgcgcagagaacgcc

SEQ ID NO: 23 - DNA, 39 bp, Artificial Sequence
  Primer JPO003 F 39
cgtcccacctccaagtctgccaatctgacgttcttcatc

SEQ ID NO: 24 - DNA, 39 bp, Artificial Sequence
  Primer JPO003 R 39
agacttggaggtgggacgcttcgccgccgtcagctgtcc

SEQ ID NO: 25 - protein, 21 aa, Artificial Sequence
  JPO002 signal peptide
MRLTLLSGVAGVLCAGKRTGL

SEQ ID NO: 26 - protein, 23 aa, Artificial Sequence
  JPO003 signal peptide
MRLTLLSGVAGVLCAGQLTAAAK

SEQ ID NO: 27 - DNA, 49 bp, Artificial Sequence
  HTJP-1022 forward primer for the JSP001 and amylase
ttgaggatttagtcttgatcggatccaccatgcggctctccacatcctc

SEQ ID NO: 28 - DNA, 48 bp, Artificial Sequence
  HTJP-1049 reverse primer for the JSP001 and amylase
ctatgcgttatcgtacgcaccacgtgctaccgccaggtgtcagtcacc

SEQ ID NO: 29 - DNA, 88 bp, Artificial Sequence
  HTJP-1053, JSP035 template DNA1
attcttcaaaattgaggatttagtcttgatcggatccaccatgcggctctccacatcctccctcttcttgtccgtctccttgctcgga

SEQ ID NO: 30 - DNA, 115 bp, Artificial Sequence
  HTJP-1149, JSP035 template DNA2
tccctcttcttgtccgtctccttgctcggaaagttggccttgggcttcgcacgtgcacctgttgctgctagagctgtaagtaacatccactctgttctagtgccatgctgagatt

SEQ ID NO: 31 - DNA, 45 bp, Artificial Sequence
  HTJP-1183, Forward primer for first PCR (JSP035)
gatttagtcttgatcggatccaccatgcggctctccacatcctcc

SEQ ID NO: 32 - DNA, 40 bp, Artificial Sequence
  HTJP-1184, Reverse primer for 1st PCR (JSP035 and JSP038)
ttccaatcgtccgacgtcgcagctctagcagcaacaggtg

SEQ ID NO: 33 - DNA, 86 bp, Artificial Sequence
  HTJP-1112, JSP038 Template DNA1
attcttcaaaattgaggatttagtcttgatcggatccaccatgggtgtctctgccgttctacttcctttgtacctcctgtccggag

SEQ ID NO: 34 - DNA, 117 bp, Artificial Sequence
  HTJP-1151, JSP038 Template DNA2
ttctacttcctttgtacctcctgtccggagttaccttcggactggcattcgcacgtgcacctgttgctgctagagctgtaagtaacatccactctgttctagtgccatgctgagatt

SEQ ID NO: 35 - DNA, 49 bp, Artificial Sequence
  HTJP-1187, Forward primer for 1st PCR (JSP038)
gatttagtcttgatcggatccaccatgggtgtctctgccgttctacttc

SEQ ID NO: 36 - DNA, 50 bp, Artificial Sequence
  HTJP-1185, Forward primer for 1st PCR (JSP035)
ggcttcgcacgtgcacctgttgctgctagagctgcgacgtcggacgattg

SEQ ID NO: 37 - DNA, 50 bp, Artificial Sequence
  HTJP-1186, Forward primer for 1st PCR (JSP038)
gtgcacctgttgctgctagagctgcgacgtcggacgattggaagggtaag

SEQ ID NO: 38 - DNA, 20 bp, Artificial Sequence
  HTJP-324, Primer for the southern probe in the promoter region
aagggatgcaagaccaaacc

SEQ ID NO: 39 - DNA, 22 bp, Artificial Sequence
  HTJP-325, Primer for the southern probe in the promoter region
tgaagaatttgtgttgtctgag

SEQ ID NO: 40 - DNA, 63 bp, Artificial Sequence
  JSP001 (control), Nucleotide sequence of signal peptide
atgcggctctccacatcctccctcttcttgtccgtctccttgctcggaaagttggccttgggc

SEQ ID NO: 41 - protein, 21 aa, Artificial Sequence
  JSP001, Amino acid sequence of signal peptide
MRLSTSSLFLSVSLLGKLALG

SEQ ID NO: 42 - DNA, 90 bp, Artificial Sequence
  JSP035, Nucleotide sequence of signal peptide with leader peptide
atgcggctctccacatcctccctcttcttgtccgtctccttgctcggaaagttggccttgggcttcgcacgtgcacctgttgctgctaga

SEQ ID NO: 43 - protein, 30 aa, Artificial Sequence
  JSP035, Amino acid sequence of signal peptide with leader peptide
MRLSTSSLFLSVSLLGKLALGFARAPVAAR

SEQ ID NO: 44 - DNA, 90 bp, Artificial Sequence
  JSP038, Nucleotide sequence of signal peptide with leader peptide
atgggtgtctctgccgttctacttcctttgtacctcctgtccggagttaccttcggactggcattcgcacgtgcacctgttgctgctaga

SEQ ID NO: 45 - protein, 30 aa, Artificial Sequence
  JSP038, Amino acid sequence of signal peptide with leader peptide
MGVSAVLLPLYLLSGVTFGLAFARAPVAAR

SEQ ID NO: 46 - DNA, 1815 bp, Artificial Sequence
  JSP001 , Nucleotide sequence of signal peptide with leader peptide and alpha-amylase
atgcggctctccacatcctccctcttcttgtccgtctccttgctcggaaagttggccttgggcgcgacgtcggacgattggaagggtaaggccatttaccagttgctcacggaccgattcggtcgcgcagatgactcgacctcgaactgttcgaacctctcgaactactgtggtggcacttacgagggcatcactaaacatctcgactacatctccggtatgggcttcgatgcaatttggatttcgccgatccctaagaactcggacggtggataccacggttactgggccacagacttctatcagctcaactcgaacttcggcgacgagtcgcagttgaaagcgctcatccaggcggcccatgagcgggacatgtatgtcatgctcgatgtggtggcaaaccacgccggcccgacttcgaacggatactcgggttacactttcgacgatgcctccctctaccatccgaaatgtaccatcgattacaacaaccagacatcgatcgaacagtgttgggtcgccgatgagttgcccgatatcgacaccgaaaactcggacaacgtcgcaatcctcaacgacatcgtctccggctgggtgggtaactactcgttcgatggtattcggatcgacaccgtcaagcacatccgcaaggacttctggacaggttacgccgaagccgcgggtgtgttcgcgaccggagaggtgttcaacggagaccccgcatacgtgggaccctatcagaaatacttgccttccctcatcaactatcccatgtactacgccctcaacgacgtcttcgtctcgaagtcgaagggtttctccaggatttccgagatgttgggctcgaaccgtaacgccttcgaagatacttccgtcctcaccacgttcgtggacaaccacgacaaccctcgattcttgaactcccagtccgacaaagccctcttcaagaacgcgctcacatacgtgttgctcggcgaaggaatccccatcgtctactatggatcggaacagggcttctcgggcggtgcagaccctgccaaccgagaagtcctctggactacgaactacgacacgtcgtcggatctctaccagttcatcaagaccgtcaactcggtgcgtatgaagtcgaacaaggcggtgtacatggacatttacgtgggcgataacgcgtatgcattcaagcatggagacgccttggtggtcctcaacaactacggctcgggttcgaccaaccaggtgtccttctcggtgtcgggaaagttcgactccggcgcctccctcatggatatcgtgtccaacatcacaactactgtctcctcggatggcacagtcactttcaacttgaaggatggcctcccggcgattttcacctccgcaactggcggcaccactacgacggctacccccactggctccggcagcgtgacctcgaccagcaagaccaccgcgactgccagcaagaccagcaccagtacgtcatcaacctcctgtaccactcccaccgccgtggctgtgactttcgatctgacagctaccaccacctacggcgagaacatctacctggtcggatcgatctctcagctgggtgactgggaaaccagcgacggcatagctctgagtgctgacaagtacacttccagcgacccgctctggtatgtcactgtgactctgccggctggtgagtcgtttgagtacaagtttatccgcattgagagcgatgactccgtggagtgggagagtgatcccaaccgagaatacaccgttcctcaggcgtgcggaacgtcgaccgcgacggtgactgacacctggcggtag

SEQ ID NO: 47 - protein, 604 aa, Artificial Sequence
  JSP001, Amino acid sequence of signal peptide with leader peptide and alpha-amylase
MRLSTSSLFLSVSLLGKLALGATSDDWKGKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFDDASLYHPKCTIDYNNQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 48 - DNA, 1845 bp, Artificial Sequence
  JSP035, Nucleotide sequence of signal peptide with leader peptide and alpha-amylase
atgcggctctccacatcctccctcttcttgtccgtctccttgctcggaaagttggccttgggcttcgcacgtgcacctgttgctgctagagctgcgacgtcggacgattggaagggtaaggccatttaccagttgctcacggaccgattcggtcgcgcagatgactcgacctcgaactgttcgaacctctcgaactactgtggtggcacttacgagggcatcactaaacatctcgactacatctccggtatgggcttcgatgcaatttggatttcgccgatccctaagaactcggacggtggataccacggttactgggccacagacttctatcagctcaactcgaacttcggcgacgagtcgcagttgaaagcgctcatccaggcggcccatgagcgggacatgtatgtcatgctcgatgtggtggcaaaccacgccggcccgacttcgaacggatactcgggttacactttcgatgatgcctccctctaccatccgaaatgtaccatcgattacaacaaccagacatcgatcgaacagtgttgggtcgccgatgagttgcccgatatcgacaccgaaaactcggacaacgtcgcaatcctcaacgacatcgtctccggctgggtgggtaactactcgttcgatggtattcggatcgacaccgtcaagcacatccgcaaggacttctggacaggttacgccgaagccgcgggtgtgttcgcgaccggagaggtgttcaacggagaccccgcatacgtgggaccctatcagaaatacttgccttccctcatcaactatcccatgtactacgccctcaacgacgtcttcgtctcgaagtcgaagggtttctccaggatttccgagatgttgggctcgaaccgtaacgccttcgaagatacttccgtcctcaccacgttcgtggacaaccacgacaaccctcgattcttgaactcccagtccgacaaagccctcttcaagaacgcgctcacatacgtgttgctcggcgaaggaatccccatcgtctactatggatcggaacagggcttctcgggcggtgcagaccctgccaaccgagaagtcctctggactacgaactacgacacgtcgtcggatctctaccagttcatcaagaccgtcaactcggtgcgtatgaagtcgaacaaggcggtgtacatggacatttacgtgggcgataacgcgtatgcattcaagcatggagacgccttggtggtcctcaacaactacggctcgggttcgaccaaccaggtgtccttctcggtgtcgggaaagttcgactccggcgcctccctcatggatatcgtgtccaacatcacaactactgtctcctcggatggcacagtcactttcaacttgaaggatggcctcccggcgattttcacctccgcaactggcggcaccactacgacggctacccccactggctccggcagcgtgacctcgaccagcaagaccaccgcgactgccagcaagaccagcaccagtacgtcatcaacctcctgtaccactcccaccgccgtggctgtgactttcgatctgacagctaccaccacctacggcgagaacatctacctggtcggatcgatctctcagctgggtgactgggaaaccagcgacggcatagctctgagtgctgacaagtacacttccagcgacccgctctggtatgtcactgtgactctgccggctggtgagtcgtttgagtacaagtttatccgcattgagagcgatgactccgtggagtgggagagtgatcccaaccgagaatacaccgttcctcaggcgtgcggaacgtcgaccgcgacggtgactgacacctggcggtag

SEQ ID NO: 49 - protein, 614 aa, Artificial Sequence
  JSP035, Amino acid sequence of signal peptide with leader peptide and alpha-amylase
MRLSTSSLFLSVSLLGKLALGFARAPVAARAATSDDWKGKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFDDASLYHPKCTIDYNNQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 50 - DNA, 1845 bp, Artificial Sequence
  JSP038, Nucleotide sequence of signal peptide with leader peptide and alpha-amylase
atgggtgtctctgccgttctacttcctttgtacctcctgtccggagttaccttcggactggcattcgcacgtgcacctgttgctgctagagctgcgacgtcggacgattggaagggtaaggccatttaccagttgctcacggaccgattcggtcgcgcagatgactcgacctcgaactgttcgaacctctcgaactactgtggtggcacttacgagggcatcactaaacatctcgactacatctccggtatgggcttcgatgcaatttggatttcgccgatccctaagaactcggacggtggataccacggttactgggccacagacttctatcagctcaactcgaacttcggcgacgagtcgcagttgaaagcgctcatccaggcggcccatgagcgggacatgtatgtcatgctcgatgtggtggcaaaccacgccggcccgacttcgaacggatactcgggttacactttcgatgatgcctccctctaccatccgaaatgtaccatcgattacaacaaccagacatcgatcgaacagtgttgggtcgccgatgagttgcccgatatcgacaccgaaaactcggacaacgtcgcaatcctcaacgacatcgtctccggctgggtgggtaactactcgttcgatggtattcggatcgacaccgtcaagcacatccgcaaggacttctggacaggttacgccgaagccgcgggtgtgttcgcgaccggagaggtgttcaacggagaccccgcatacgtgggaccctatcagaaatacttgccttccctcatcaactatcccatgtactacgccctcaacgacgtcttcgtctcgaagtcgaagggtttctccaggatttccgagatgttgggctcgaaccgtaacgccttcgaagatacttccgtcctcaccacgttcgtggacaaccacgacaaccctcgattcttgaactcccagtccgacaaagccctcttcaagaacgcgctcacatacgtgttgctcggcgaaggaatccccatcgtctactatggatcggaacagggcttctcgggcggtgcagaccctgccaaccgagaagtcctctggactacgaactacgacacgtcgtcggatctctaccagttcatcaagaccgtcaactcggtgcgtatgaagtcgaacaaggcggtgtacatggacatttacgtgggcgataacgcgtatgcattcaagcatggagacgccttggtggtcctcaacaactacggctcgggttcgaccaaccaggtgtccttctcggtgtcgggaaagttcgactccggcgcctccctcatggatatcgtgtccaacatcacaactactgtctcctcggatggcacagtcactttcaacttgaaggatggcctcccggcgattttcacctccgcaactggcggcaccactacgacggctacccccactggctccggcagcgtgacctcgaccagcaagaccaccgcgactgccagcaagaccagcaccagtacgtcatcaacctcctgtaccactcccaccgccgtggctgtgactttcgatctgacagctaccaccacctacggcgagaacatctacctggtcggatcgatctctcagctgggtgactgggaaaccagcgacggcatagctctgagtgctgacaagtacacttccagcgacccgctctggtatgtcactgtgactctgccggctggtgagtcgtttgagtacaagtttatccgcattgagagcgatgactccgtggagtgggagagtgatcccaaccgagaatacaccgttcctcaggcgtgcggaacgtcgaccgcgacggtgactgacacctggcggtag

SEQ ID NO: 51 - protein, 614 aa, Artificial Sequence
  JSP038, Amino acid sequence of signal peptide with leader peptide and alpha-amylase
MGVSAVLLPLYLLSGVTFGLAFARAPVAARAATSDDWKGKAIYQLLTDRFGRADDSTSNCSNLSNYCGGTYEGITKHLDYISGMGFDAIWISPIPKNSDGGYHGYWATDFYQLNSNFGDESQLKALIQAAHERDMYVMLDVVANHAGPTSNGYSGYTFDDASLYHPKCTIDYNNQTSIEQCWVADELPDIDTENSDNVAILNDIVSGWVGNYSFDGIRIDTVKHIRKDFWTGYAEAAGVFATGEVFNGDPAYVGPYQKYLPSLINYPMYYALNDVFVSKSKGFSRISEMLGSNRNAFEDTSVLTTFVDNHDNPRFLNSQSDKALFKNALTYVLLGEGIPIVYYGSEQGFSGGADPANREVLWTTNYDTSSDLYQFIKTVNSVRMKSNKAVYMDIYVGDNAYAFKHGDALVVLNNYGSGSTNQVSFSVSGKFDSGASLMDIVSNITTTVSSDGTVTFNLKDGLPAIFTSATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 52 - protein, 21 aa, Artificial Sequence
  JSP038, Amino acid sequence of signal peptide
MGVSAVLLPLYLLSGVTFGLA